(19) United States
(12) Patent Application Publication
(10) Pub. No.: US 2012/0156311 A1
(43) Pub. Date: Jun. 21, 2012

(54) COMBINATION THERAPY FOR CANCER COMPRISING A PLATINUM-BASED ANTINEOPLASTIC AGENT AND A BIOCOMPATIBLE ELECTRON DONOR

(76) Inventor: Qing-Bin Lu, Waterloo (CA)

(21) Appl. No.: 13/393,284

(22) PCT Filed: Sep. 1, 2010
(86) PCT No.: PCT/CA2010/001327

§ 371 Date: Feb. 29, 2012

Related U.S. Application Data
(60) Provisional application No. 61/272,208, filed on Sep. 1, 2009.
(60) Provisional application No. 61/272,479, filed on Sep. 29, 2009.
(60) Provisional application No. 61/344,064, filed on May 17, 2010.

(51) Int. Cl. A61K 33/24; C07C 211/51; A61K 35/00; C07D 403/06

(52) U.S. Cl. 424/649; 548/427; 564/305

(57) ABSTRACT

The combination of a biocompatible electron donor and a platinum-based antineoplastic agent exhibits improved efficacy in treating cancer This improved activity appears to be the result of electron transfer from the aforementioned donor compound to the platinum-based antineoplastic agent As the electron donor alone has no chemotherapeutic utility in treating cancer, the resulting combinations appear to be synergistic in nature In select preferred embodiments, the biocompatible electron donor is an amine (such as N,N,N′,N′-tetramethyl-p-phenylene diamine or indocyanine green), a phenolic compound (such as a flavanol or catechin), or a quinone (such as an aromatic quinone), while the antineoplastic is cisplatin.

### CROSS REFERENCE TO RELATED APPLICATIONS

This application claims the benefit of priority of U.S. Provisional Patent Application No. 61/272,208 filed Sep. 1, 2009, U.S. Provisional Patent Application No. 61/272,479 filed Sep. 29, 2009, and U.S. Provisional Patent Application No. 61/344,064 filed May 17, 2010, each of which is incorporated herein by reference in its entirety.

### FIELD OF THE DISCLOSURE

The present disclosure relates to a combination treatment for cancer comprising a platinum-based antineoplastic agent and a biocompatible electron donor.

### BACKGROUND

Current cancer therapy generally involves treatment with surgery, radiation therapy, chemotherapy, or a combination of these approaches, each of which has significant drawbacks and limitations.

Chemotherapy generally refers to the treatment of cancer with one or more antineoplastic (e.g. anticancer) agents. Many antineoplastic agents act by impairing mitosis and thereby target rapidly-dividing cells, a hallmark property of most cancer cells. Some agents stop the cells from dividing and others kill the cells, e.g. by triggering apoptosis. Certain newer agents are being developed to provide a more targeted therapy, for example, those targeting specific proteins expressed on cancer cells. Some antineoplastic agents, such as bioreductive agents, become preferentially active at the site of a tumor, e.g. under hypoxic conditions.

While a variety of antineoplastic agents are available, nearly all are toxic. Chemotherapy generally causes significant, and often dangerous, side effects, including severe nausea and vomiting, bone marrow depression, immunosuppression, cytopenia, pain and fatigue. Additional side-effects can include cachexia, mucositis, alopecia, cutaneous complications, such as hypersensitivity reactions, as well as neurological, pulmonary, cardiac, reproductive and endocrine complications. Side effects associated with antineoplastic agents are generally the major factor in defining a dose-limiting toxicity (DLT) for the agent. Managing the adverse side effects induced by chemotherapy is of major importance in the clinical management of cancer treatment. In addition, many tumor cells are resistant, or develop resistance, to antineoplastic agents, e.g. through multi-drug resistance.

Combination therapeutic approaches that permit the use of lower doses of antineoplastic agents than doses conventionally used in monotherapy, while maintaining anticancer efficacy, are highly desirable. In many combinations, there is no synergistic interaction between the combined agents, for instance, the two agents may act via different mechanisms of action, or target different pathways, thereby achieving an additive therapeutic effect while reducing the side effects associated with each individual agent. In some combinations, a synergistic effect may be achieved between two or more agents, where the combined effect is greater than the additive effect of the individual agents. For example, a beneficial interaction between the agents may occur in vivo. Synergistic combinations are desirable but rare. In some combination therapies, each agent administered exerts therapeutic effects. However, in other combination therapies, a therapeutic agent may be administered in combination with a non-therapeutic agent that directly or indirectly enhances the activity of the therapeutic agent or otherwise modifies its effects in a beneficial manner.

Effective combination therapies, including anticancer therapies, are desirable, since they can lead to a decrease in the frequency and/or severity of adverse side effects and an improved quality of life for the patient. Benefits of reducing the incidence of side effects include improved patient compliance, a reduction in the number of hospitalizations needed for the treatment of adverse effects, and a decrease in the administration of analgesic agents needed to treat pain associated with the adverse effects. Where dose-limiting toxicity is not an issue, combination therapy can also maximize the therapeutic effects of antineoplastic agents administered at higher doses. In addition to increased anticancer efficacy, such approaches may reduce or overcome the development of resistance.

The identification of new antineoplastic agents remains a somewhat empirical process, generally involving screening a large number of compounds in order to identify a very small number of potential candidate molecules for further investigation. Thus, there is a need for a more rational and efficient approach to the design of novel antineoplastic agents. The same applies for the development of novel combinations of agents for enhanced chemotherapy. While various drug discovery tools are available, such as binding-based screening, inhibitor-based screening and structure-based drug design, a major hindrance has been lack of understanding of the precise molecular mechanisms of action of most anticancer drugs currently in use or in clinical trials. Without a specific mechanistic understanding, it is difficult to learn from the successes and failures of individual therapies. Furthermore, the development of successful combination therapies, particularly ones where synergy may be achieved, is effectively reduced to trial and error. Thus, there is a need for enhanced mechanistic understanding of anticancer drug action and subsequent rational design of effective anticancer therapies, including combination therapies.

### SUMMARY

The present disclosure relates to a treatment for cancer comprising a combination of a platinum-based antineoplastic agent and a biocompatible electron donor.

The present inventor has revealed the molecular mechanisms of action of cisplatin in inducing DNA damage and in combination with radiotherapy. The inventor then hypothesized that the cytotoxic activity of platinum-based anticancer agents could be enhanced in the presence of a biocompatible molecule capable of donating one or more electrons to the anticancer agent. It has now been demonstrated that contacting cancer cells with a platinum-based antineoplastic agent in the presence of a biocompatible electron donor in vitro or in vivo provides an enhanced anticancer effect.

In a first aspect, the present disclosure provides a method for the treatment of a cancer. The method comprises administering to a subject in need thereof a therapeutically effective amount of a platinum-based antineoplastic agent, and a biocompatible electron donor. In preferred embodiments, the biocompatible electron donor is capable of transferring one or more electrons to the platinum-based antineoplastic agent to thereby synergistically enhance its anticancer effect.

In another aspect, the present disclosure provides a synergistic combination comprising a platinum-based antineoplastic agent and a biocompatible electron donor for use in the treatment of cancer.

In another aspect, the present disclosure provides a synergistic combination comprising a platinum-based antineoplastic agent and a biocompatible electron donor for use in the manufacture of a medicament for the treatment of cancer.

In another aspect, the present disclosure provides a use of a synergistic combination of a platinum-based antineoplastic agent and a biocompatible electron donor in the treatment of cancer.

In another aspect, the present disclosure provides a use of a synergistic combination of a platinum-based antineoplastic agent and a biocompatible electron donor in the manufacture of a medicament for the treatment of cancer.

In another aspect, the present disclosure provides a kit or commercial package comprising a biocompatible electron donor and a platinum-based antineoplastic agent, together with instructions for carrying out a combination therapy for the treatment of a cancer. In some embodiments, the biocompatible electron donor and the platinum-based antineoplastic agent are in separate pharmaceutical compositions.

In another aspect, the present disclosure provides a biocompatible electron donor for use in combination with a platinum-based antineoplastic agent for the treatment of cancer.

Exemplary embodiments of the method, combination, composition, use, commercial package, kit or biocompatible electron donor are described herein.

In some embodiments, the biocompatible electron donor comprises one or more atoms having a lone electron pair selected from the group consisting of O, N or S. For example, the lone electron pair may be present in a heteroaryl ring or a heterocyclic ring. In some embodiments, the one or more atoms having a lone electron pair is present in an electron-donating substituent. The electron-donating substituent may be, for example, —O, —OR, —OH, —SR, —SH, —NH₂, —NHR, or —NR₁R₂, —NHCOCH₃, —NHCOR, —OCH₃. R, R₁ and R₂ can be the same or different, and may be selected from the group consisting of substituted or unsubstituted alkyl, alkenyl, alkynyl, aryl, and aralkyl. In some embodiments, the electron-donating substituent is —NH₂, —NHR, or —NR₁R₂. In some embodiments, the electron-donating substituent is coupled to a structure capable of stabilizing a charge following donation of an electron. In one embodiment, the electron-donating group is NR¹R₂ and wherein R₁ and R₂ are each methyl.

In some embodiments, the biocompatible electron donor is capable of donating two or more electrons. In some embodiments, the biocompatible electron donor is capable of donating two electrons.

In some embodiments, the biocompatible electron donor is selected from the group consisting of amine compounds; phenolic compounds; and quinones.

In some embodiments, the biocompatible electron donor is an amine compound comprising two nitrogen atoms having a lone electron pair, and further comprising alkyl substituents that increase the basicity of the nitrogen atoms.

In some embodiments, the biocompatible electron donor is N,N,N′,N′-tetramethyl-p-phenylene diamine or indocyanine green.

In some embodiments, the biocompatible electron donor is a phenolic compound, such as a phenol or polyphenol, in particular, a flavanol (catechins), such as epigallocatechin gallate (EGCG), epigallocatechin (EGC), epicatechin gallate (ECG) or epicatechin (EC).

In some embodiments, the biocompatible electron donor is a quinone, such as benzoquinone, naphthoquinone or anthraquinone.

In some embodiments, the platinum-based antineoplastic agent is selected from the group consisting of cisplatin, carboplatin, nedaplatin, oxaliplatin, satraplatin, and triplatin tetranitrate. In some embodiments, the platinum-based antineoplastic agent is cisplatin.

In therapeutic embodiments, the platinum-based antineoplastic agent and the biocompatible electron donor, or the combination, are administered (or, are for administration) in a therapeutically effective amount.

In some embodiments, the platinum-based antineoplastic agent and the biocompatible electron donor are administered (or, are for administration) simultaneously or sequentially. In some embodiments, the platinum-based antineoplastic agent and the biocompatible electron donor are administered (or, are for administration) sequentially.

In some embodiments, the biocompatible electron donor and the platinum-based antineoplastic agent are administered (or, are for administration) parenterally. In some embodiments, the parenteral administration is systemic or regional. In some embodiments, the parenteral administration is intravenous, intraarterial or intraperitoneal.

In some embodiments, the biocompatible electron donor is administered (or, is for administration) in excess of the platinum-based antineoplastic agent.

In some embodiments, the cancer is testicular cancer, bladder cancer, cervical cancer, ovarian cancer, breast cancer, prostate cancer, head cancer, neck cancer, or lung cancer (e.g. non small cell lung cancer).

In some embodiments, the platinum-based antineoplastic agent is cisplatin and the biocompatible electron donor is TPMD or ICG. In some embodiments, the platinum-based antineoplastic agent is cisplatin and the biocompatible electron donor is TPMD. In some embodiments, the platinum-based antineoplastic agent is cisplatin and the biocompatible electron donor is ICG.

Some potential advantages to be achieved by the present disclosure include the development of highly effective chemotherapies with fewer toxic side effects, reduced drug resistance, better targeting at tumor cells, and broader clinical applications of platinum-based antineoplastic agents to various tumors, such as cervical, ovarian, breast and prostate cancers.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 illustrates cell survival rates of HeLa (cervical cancer) cells after treatment with ICG alone. ICG is an exemplary biocompatible electron donor. The viability of cells in 96-well plates was measured by MTT assay.

FIG. 2 illustrates cell survival rates of HeLa (cervical cancer) cells after treatment with TMPD alone. TMPD is an exemplary biocompatible electron donor. The viability of cells in 96-well plates was measured by MTT assay.

FIG. 3 illustrates cell survival rates of HeLa (cervical cancer) cells after treatment with the combination of 15 μM cisplatin (CDDP) with various concentrations of ICG (A); various concentrations of cisplatin alone (B, hatched lines); and the combination of 100 μM ICG with various concentrations of cisplatin (B, solid black).

FIG. 4 illustrates cell survival rates of HeLa (cervical cancer) cells after treatment with various concentrations of cisplatin alone (hatched lines), and the combination of cisplatin with 100 uM TMPD (solid black). TMPD significantly reduced the cisplatin dose required to kill cancer cells.

FIG. 5 shows results of an APO-BrdU DNA Fragmentation Assay for HeLa (cervical cancer) cells: (a) untreated; (b) treated with 100 μM TMPD alone; (c) treated with 25 μM CDDP alone; and (d) 25 μM CDDP+100 μM TMPD for 48 h. The density plot illustrates BrdU-positive cells (DNA fragmentation) as a function of the DNA content (position in the cell cycle). TMPD increased the DNA fragmentation of HeLa cells treated with cisplatin from 6% to 69%.

FIG. 6 illustrates cell survival rates of NIH-OVCAR-3 (HTB-161) cisplatin-resistant human ovarian cancer cells after treatment with various concentrations of cisplatin alone (hatched lines) and combined cisplatin with 100 μM TMPD (solid black). TMPD significantly reduced the cisplatin dose required to kill cancer cells and advantageously circumvented the drug resistance of these cancer cells.

FIG. 7 shows DNA fragmentation measurements of NIH-OVCAR-3 (HTB-161) cisplatin-resistant human ovarian cancer cells with treatment of 100 μM TMPD alone, 50 μM cisplatin alone, and 50 μM cisplatin plus 100 μM TMPD. TPMD significantly increased the DNA fragmentation of HTB-161 cells from 3.61% to 16.43%.

FIG. 8 shows representative micrographs of NIH-OVCAR-3 (HTB-161) cisplatin-resistant human ovarian cancer cells undergoing apoptosis induced by treatment with 100 μM TMPD, 50 μM CDDP, or 50 μM CDDP plus 100 μM TMPD, for 10 h as assessed by fluorescence microscopy. The images at the left are the cell nuclei detected by blue fluorescence of Hoechest 33342 staining, while those at the right are apoptotic cells detected by green fluorescence of FLICA reagent representing caspase activation. The combination of TMPD and cisplatin significantly increased the population of apoptotic cells, as can be clearly seen in the representative black & white images.

FIG. 9 (Upper) shows agarose gel electrophoresis images of plasmid DNA in control (100 μM cisplatin), and 100 μM TMPD combined with 25, 50 or 100 μM cisplatin; FIG. 9 (lower) shows Densitograms of the gel image. TMPD significantly increased the yield of DNA double-strand breaks (DSBs).

FIG. 10 illustrates the results of γH2AX in HeLa cells treated with cisplatin alone and cisplatin plus 100 μM TMPD. The γH2AX intensity is proportional to the yield of DNA double-strand breaks (DSBs).

FIG. 11 shows tumor volume growth curves for the treatments of control, cisplatin only, cisplatin plus ICG, and cisplatin plus TMPD in a murine breast tumor model. The tumor volumes are normalized to volumes just prior to drug treatment (i.e., at Day 1).

FIG. 12 shows mouse weight variation for the treatments of control, cisplatin only, cisplatin plus ICG, cisplatin plus TMPD. The mouse weights are normalized to those before drug treatment.

### DETAILED DESCRIPTION

Generally, the present disclosure relates to a combination therapy for cancer.

More particularly, the present disclosure relates to a combination therapy for cancer comprising a platinum-based antineoplastic agent and a biocompatible electron donor. It is demonstrated herein that contacting cancer cells with a platinum-based antineoplastic agent in the presence of an electron-donating compound in vitro or in vivo provides an enhanced anticancer effect of the antineoplastic agent, i.e. synergy.

Disclosed herein are compounds, compositions, methods, uses, commercial packages and kits relating to the combination therapy.

### Platinum-Based Antineoplastic Agents

Platinum-based antineoplastic agents, sometimes called platinum analogues, coordinate to DNA, binding predominantly to N7 of guanine bases, to interfere with DNA repair. They act in a manner similar to traditional alkylating agents in this respect, except that they do not possess an alkyl group. Thus, they are often referred to as “alkylating-like” antineoplastic agents. Examples include, but are not limited to, cisplatin, carboplatin, nedaplatin, oxaliplatin, satraplatin, and triplatin tetranitrate.

Cisplatin (cis-diamminedichloroplatinum(II) or CDDP), having the molecular formula cis-Pt(NH3)₂Cl₂, remains one of the most widely used drugs for cancer treatment, including sarcomas, some carcinomas, lymphomas and germ cell tumors. It is particularly effective in treating testicular, bladder, and ovarian cancers, and is increasingly used against cervical, head, neck, and non small cell lung cancers [1-3]. Like many antineoplastic drugs, cisplatin is generally administered intravenously, and is generally dissolved in a saline solution. Administration time is typically about 30 minutes to 2 hours per treatment at an infusion rate of about 1 mg/minute. Cisplatin is sometimes given by intraarterial and intraperitoneal routes as well. For instance, intraarterial administration is often used for melanoma, glioblastoma, and liver cancer. For some cancers (e.g. ovarian cancer), cisplatin is administered into a bodily cavity rather than a blood vessel. The type and extent of a cancer determines the exact dose and schedule of administering of this drug.

Despite its widespread use, cisplatin has two major drawbacks: severe toxic side effects and both intrinsic and acquired resistance. These drawbacks currently limit the clinical applications of cisplatin to many common human cancers [4]. Research is underway to identify less toxic analogues and to develop combination therapies that reduce cisplatin toxicity and prevent or overcome drug resistance. Over the past 40 years, over 3000 cisplatin analogues have been synthesized and tested [1-3]. One such analogue, oxaliplatin, has now been approved by the FDA for the treatment of colorectal cancer [3]. Transplatin, the trans stereoisomer of cisplatin, does not exhibit a comparably useful pharmacological effect and is toxic, thus it is desirable to test batches of cisplatin for the absence of the trans isomer.

### Dissociative-Electron-Transfer Mechanism of Cisplatin

Understanding the precise molecular mechanisms that induce DNA damage/repair and cell death should provide a better understanding of the causes of cancer and also foster the development of improved treatment strategies. Oxidative molecular pathways leading to DNA damage and cell death are fairly well studied in relation to human cancers and cancer treatment. However, very little is known about the role of reductive molecular pathways in cancer and cancer treatment.

The present inventor has initiated a molecular-mechanism-based drug discovery program, in particular, applying unique femtosecond time-resolved laser spectroscopic (fs-TRLS) techniques to deduce the precise molecular mechanisms of action of drugs. Knowledge gained in this emerging transdisciplinary frontier of ‘femtomedicine’ (a term coined by the inventor) holds the promise of major advances in cancer therapy.

The present inventor has recently deduced the molecular mechanisms of action of cisplatin in inducing DNA damage and in combination with radiotherapy [5a and b]. Although cisplatin is a well-known DNA-attacking agent, its precise molecular mechanism of action had remained elusive until recently solved [5, 6], as described below. The inventor further hypothesized that the anticancer activity of platinum-based antineoplastic agents, such as cisplatin and its analogues and derivatives, could be enhanced in the presence of a biocompatible molecule capable of donating one or more electrons to the antineoplastic agent. This hypothesis is demonstrated herein to be true.

Applying the principles disclosed herein, persons of skill in the art will be able to identify electron-donating compounds that can enhance the anticancer activity of platinum-based antineoplastic agents. Thus, the scope of the present disclosure extends beyond the exemplary compounds and combinations disclosed.

By way of background, femtosecond time-resolved laser spectroscopy (fs-TRLS) is a versatile and powerful technique for real-time observation of molecular reactions and has been described as the world's fastest “camera”. It uses laser flashes of extremely short duration, along the time scale on which reactions actually occur, i.e. femtoseconds (fs), where 1fs=10⁻¹⁵ seconds. The “camera” records the events in a molecular reaction by initiating the reaction with a femtosecond laser pulse (pump pulse) in a certain color (wavelength). The reactant molecule is instantly excited into a higher energy state. A short time later, a second pulse (probe pulse) in a different color takes a “picture” of the reacting molecules or the newly-created species. By successively delaying the probe pulse, a “film” is obtained of the course of the reaction. The “camera” gives no direct image of the molecules. Instead, the reacting molecules or new species are observed by measuring certain characteristic properties, e.g., an optical transmission (an absorption spectrum is obtained). The one or more transition states probed (detected) at chosen wavelengths (colors) have specific spectra that serve as fingerprints, and they can therefore be identified and characterized. In the present applications, the pump pulse is used to initiate the reaction or to create a reacting species, while the probe is to detect the intermediate states during a reaction. The electronics recording the spectrum and the delay time are integrated into a labview program that directly gives rise to a transient absorption or fluorescence spectrum (as a function of delay time or wavelength) to show the real-time evolution of a particular transition state. Once intermediate species are identified, the reaction pathway can be determined. This fs-TRLS technique provides a unique capability of obtaining real-time observation and control of biochemical reactions at the molecular level.

Application of these techniques revealed a molecular explanation for why low-dose cisplatin can significantly enhance the therapeutic efficacy of radiotherapy, the reason being closely related to the high electron-transfer reactivity of cisplatin [5]. It was demonstrated that cisplatin is a very effective molecule for the dissociative-electron transfer (DET) reaction with the ultrashort-lived, weakly-bound prehydrated electron generated in radiotherapy:

eₚᵣₑ⁻+Pt(NH₃)₂Cl₂→[Pt(NH₃)₂Cl₂]*⁻→Cl⁻+Pt(NH₃)₂Cl.

eₚᵣₑ⁻+Pt(NH₃)₂Cl→[Pt(NH₃)₂Cl]*⁻→Cl⁻+Pt(NH₃)_{2.} (1)

The resultant cis-Pt(NH₃)₂ radical highly effectively leads to DNA strand breaks [5].

It was further demonstrated that, for chemotherapy, cisplatin preferentially attracts two electrons from two neighboring guanine bases in DNA, since guanine is the most favored electron donor in DNA [6]:

G+Pt(NH₃)₂Cl₂→G⁺+[Pt(NH₃)₂Cl₂]*⁻→G⁺+Pt(NH₃)₂Cl.+Cl⁻

G+Pt(NH₃)₂Cl→G⁺+[Pt(NH₃)₂Cl]*⁻→G⁺+Pt(NH₃)₂.+Cl⁻ (2).

In contrast, a weaker DET reaction of cisplatin with DNA base A, and no DET reactions with C and T, were observed. This novel DET mechanism of action has answered the long-existing question of why treatment with cisplatin results in the preferential binding of the cis-Pt(NH₃)₂ to two neighboring G bases in DNA [6]. These mechanistic understandings have great potential to improve existing cancer therapies using cisplatin and it analogues and derivatives, and to break the clinical boundaries of cisplatin for the treatment of other common cancers. These results also help explain why cisplatin, as a chemotherapeutic agent, is so effective and toxic. Moreover, this knowledge can be used for rational design of novel anticancer agents and novel combination therapies involving cisplatin and its analogues and derivatives.

### Biocompatible Electron Donors as Molecular Promoters

Based on the deduced dissociative-electron-transfer (DET) mechanism of cisplatin, described above, it was hypothesized that the DET reaction of an effective electron donor with cisplatin would promote the generation of the reactive cisplatin radical and thus enhance the cytotoxicity of cisplatin in reacting with DNA. Such molecules would essentially act as molecular promoters (PM), thereby generating “synergy”. The present inventor then set out to identify potential molecular promoters that could enhance the cytotoxicity of cisplatin at lower doses and/or overcome drug resistance, to thereby improve cisplatin therapy and possibly widen the application of cisplatin and its analogues to other cancers, such as breast and prostate cancers that tend to be insensitive to cisplatin.

Generally, the molecular promoter (PMx) is expected to react with cisplatin via a DET reaction as follows:

PMx+Pt(NH₃)₂Cl₂→PMx⁺+[Pt(NH₃)₂Cl₂]*⁻→PMx⁺+Pt(NH₃)₂Cl.+Cl⁻ (3) or

PMx+Pt(NH₃)₂Cl₂→PMx²⁺+[Pt(NH₃)₂Cl₂]²*⁻→PMx²⁺+Pt(NH₃)₂.+2Cl⁻ (4).

Thus, the molecular promoter should be capable of transferring one or more electrons to cisplatin. After electron capture, cisplatin dissociates to form one or two Cl-ions and a reactive radical that attacks DNA. It was hypothesized that the resultant Pt(NH₃)₂Cl. radical, or the Pt(NH₃)₂. radical created by reaction with a second PM molecule or a second electron-donating group on the same PM molecule, could advantageously lead to DNA strand breaks, adding to the intrastrand cross-links known to be caused by cisplatin alone. It is well known that DNA strand breaks are more difficult to repair than cross-links, the most lethal being the DNA double-strand break (DSB), which is a potent inducer of mutations and cell death.

Preferred characteristics of the molecular promoters include any one or a combination of the following: (1) effective electron donor; (2) biocompatible; (3) minimal toxicity (ideally, substantially non-toxic) at doses administered; (4) enhance the cytotoxic potential of the antineoplastic agent so that lower doses can be used; (5) capable of entering a cell and preferably nucleus; (6) target tumor cells preferentially due to the hypoxic environment in which the DET reaction occurs favorably; and/or (7) ideally reduce or overcome drug resistance to the antineoplastic agent, e.g. by promoting its reactivation.

Applying the principles of the DET reaction set forth above, several molecular promoters were rationally identified that were expected to result in synergistic effect in combination with cisplatin or other platinum-based antineoplastic agents.

Two exemplary compounds, used in biological systems, were identified and selected to test the hypotheses.

Indocyanine green (ICG), shown in Scheme 1, is a tricarbocyanine dye that has been widely used in medical imaging and diagnosis [7,8]. ICG has a molecular formula C₄₃H₄₇N₂O₆S₂Na and a molecular weight of 775. Advantageously, ICG is used as an imaging molecule for tumours. ICG has been shown to bind growth factor receptor (GFR), which assists in targeting ICG to the site of a tumour. It is typically administered in a dose of up to about 2 mg/kg for diagnostics in humans.

N,N, N′,N′-tetramethyl-p-phenylene diamine (TMPD), shown in Scheme 2, has a molecular weight of 164 and is a well-known biochemical electron donor used in biological systems [9]. TMPD is an easily-oxidized compound. It may therefore be administered in a manner that slows or prevents oxidation, such as in a particular medium or vehicle.

The two exemplary compounds, ICG and TMPD, have different chemical structures and molecular weights and thus serve to demonstrate the robustness of the principles disclosed herein.

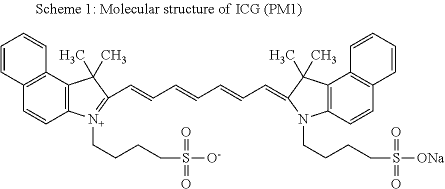

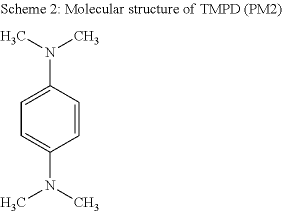

Studies were carried out to explore the in vitro and in vivo anti-tumor effects of cisplatin in combination with the two exemplary molecular promoters, ICG and TMPD, which were predicted to show synergy with cisplatin. Both compounds have two basic nitrogen (N) atoms and two alkyl (CH3) subgroups, in which the nitrogen atom features a lone electron pair and the effect of alkyl groups raises the energy of the lone pair of electrons. Such compounds can easily donate one or two electrons to activate cisplatin for chemotherapy.

### In Vitro Anticancer Effects of Combination Treatment

The in vitro anti-tumor activity of cisplatin alone, and cisplatin combined with a biocompatible electron donor, was investigated in human cervical cancers (HeLa, CCL-2) and cisplatin-resistant human ovarian cancer cells (NIH:OVCAR-3, HTB-161), as outlined in Examples 1 to 4. The HeLa cell line has been widely use in cancer research, as HeLa cells proliferate abnormally rapidly, even compared to other cancer cells [10], while the HTB-161 is an ideal model system in which to study drug resistance of clinically relevant concentrations of cisplatin and other chemotherapeutic drugs [11,12].

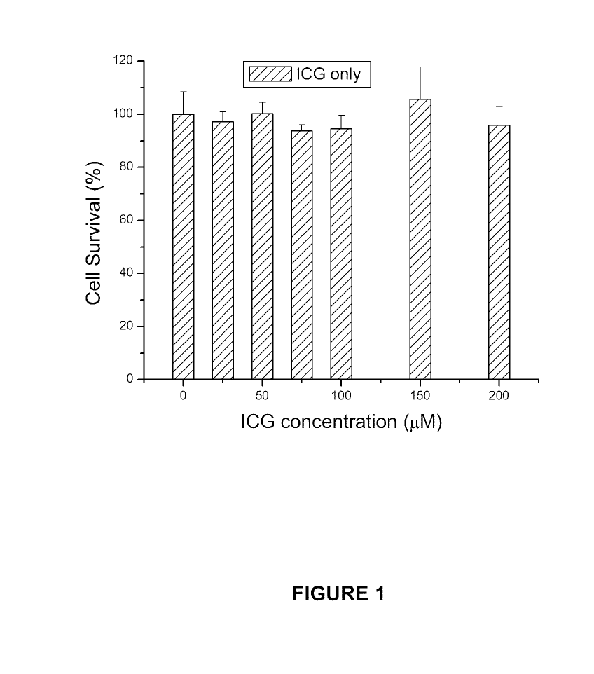

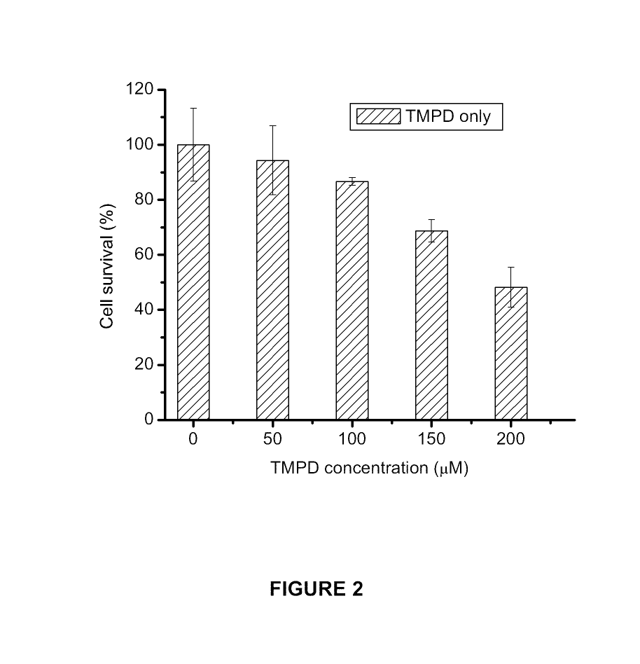

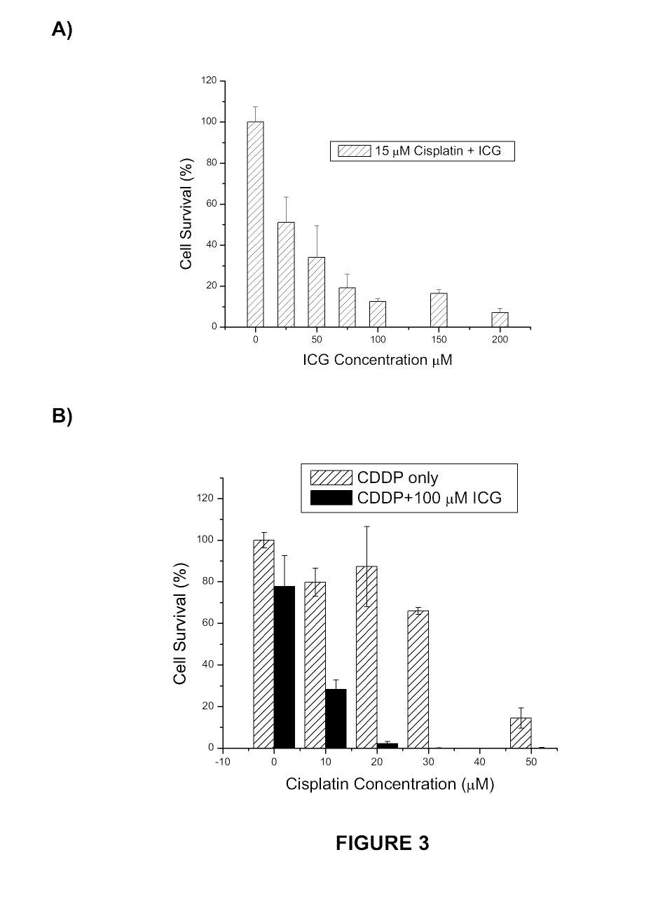

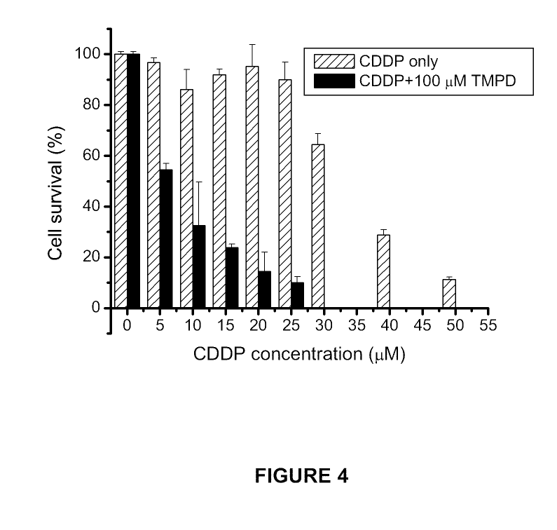

FIG. 1 and FIG. 2 illustrate cell survival rates of HeLa (cervical cancer) cells after treatment with ICG alone and TMPD alone, respectively. The results demonstrate that ICG or TMPD alone have no significant toxicity in doses up to 200 μM/100 μM, respectively. Thus, appropriate doses of the molecular promoters were selected by initially screening for toxicity. FIG. 3 illustrates cell survival rates of HeLa (cervical cancer) cells after treatment with the combination of 15 μM cisplatin with various concentrations of ICG (A); various concentrations of cisplatin (CDDP) alone (B, hatched lines); and the combination of 100 μM ICG with various concentrations of cisplatin (B, solid black). Remarkably, the addition of 100 μM (non-toxic dose) ICG to 10 and 20 μM cisplatin greatly enhanced the killing of HeLa cells from 20% to 70% and to 95%, respectively. FIG. 4 illustrates cell survival rates of HeLa (cervical cancer) cells after treatment with various concentrations of cisplatin alone (A, hatched lines), and combined cisplatin with 100 μM TMPD (A, solid black). The results demonstrate that both ICG and TMPD significantly reduce the dose of cisplatin required to kill cisplatin-sensitive tumor cells in vitro (in other words, enhance the potency of cisplatin). Thus, these molecules are expected to reduce the dose of cisplatin required to kill tumor cells in vivo, thereby reducing toxic side effects associated with the heavy-metal (Pt)-based chemotherapeutic drug in human patients. The viability of cells was measured by MTT assay, one of the most commonly used cell viability assays. This method involves the conversion of MTT (3-(4,5-dimethylthiazol-2-yl)-2,5-diphenyltetrazolium bromide) to an insoluble formazan by metabolically-active cells (live cells). Using a solubilizing agent, the formazan is dissolved and its absorbance measured, giving indication to the number of cells that survive. This is a well-established quantitative method that is rapid in terms of the required drug treatment time as well as the total protocol time compared to the clonogenic assay, which measures cell survival on the long-term scale.

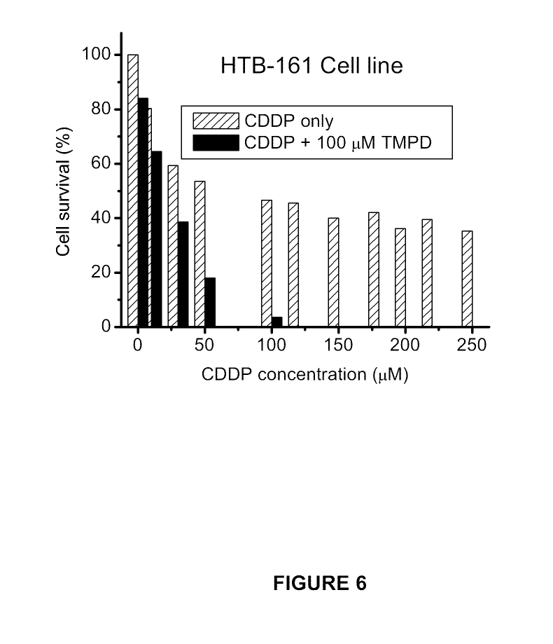

The antitumor effects of cisplatin, with and without combination with molecular promoters, was also assessed in NIH-OVCAR-3 (HTB-161) cisplatin-resistant cells. The HTB-161 has been shown to be an ideal model system to study drug resistance of clinically relevant concentrations of cisplatin and other chemotherapeutic drugs. ICG or TMPD rendered cisplatin-resistant tumor cells almost completely sensitive. FIG. 6 illustrates cell survival rates of NIH-OVCAR-3 (HTB-161) cisplatin-resistant ovarian cancer cells after treatment with various concentrations of cisplatin alone (hatched lines) and combined cisplatin with 100 uM TMPD (solid black). It was confirmed that the cisplatin-resistant HTB-161 cells indeed exhibited a strong resistance to cisplatin even at very high concentrations (200-300 uM). TMPD significantly reduced the cisplatin dose required to kill the cancer cells and, remarkably, circumvented the drug resistance of these cancer cells, demonstrating that the combination therapies disclosed herein could be effective in treating cancers that are resistant to cisplatin in vivo.

ICG and TMPD also greatly enhanced DNA fragmentation and apoptosis in cancer cells. One late stage marker of apoptosis is the degradation of DNA into small fragments. The fragmentation of DNA in apoptotic cells leads to disintegration of the nucleus. An enhancement in the amount of fragmented DNA indicates increases in apoptotic events, and thus correlates with the potency of the combination therapies. DNA fragmentation was detected using a standard apoptosis assay, APO-BrdU TUNEL, which utilizes the fact that DNA strand breaks expose a large number of 3′-OH ends, which act as a starting point for terminal deoxynucleotidyl transferase (TdT) to add deoxyribonucleotides. The break sites are labeled by the addition of BrdUTP in the presence of the TdT enzyme. An anti-BrdU antibody conjugated with a fluorescent molecule is added to detect the strand breaks by measuring their fluorescence intensities using flow cytometry. The cell cycle distribution can also be detected simultaneously from the ordinate axis of the generated dot plot (with the BrdU-content on the y-axis), where the events with fluorescence intensity above the background indicate the cells with fragmented DNA (within upper box).

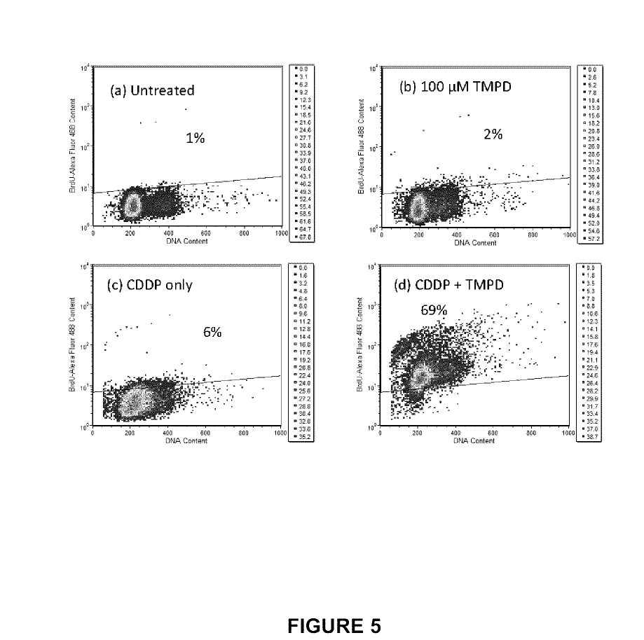

FIG. 5 shows results of an APO-BrdU DNA Fragmentation Assay for HeLa (cervical cancer) cells treated with 0, 10, 25 and 50 μM CDDP with and without 100 μM TMPD for 48 h. Density plot illustrates BrdU-positive cells (DNA fragmentation) as a function of the DNA content (position in the cell cycle). TMPD significantly increased the DNA fragmentation of HeLa cells from 6% to 69%. The APO-BrdU TUNEL assay was used to determine DNA fragmentation and cell apoptosis. As seen in FIG. 5a, only 1% of untreated cells exhibited DNA fragmentation, which can be attributed to cells naturally undergoing cell death. Most of the events were in the G1 phase of the cell cycle, characteristic of a regular cell cycle distribution. FIG. 5b shows that 2% of cells treated with 100 μM TMPD alone had DNA fragmentation. When cells were treated with 10 μM CDDP+100 μM TMPD, the percentages increased from 9% to 18% compared to cells treated with only 10 μM CDDP (not shown). Remarkably, the percentages increased from 6% to 69% when cells were treated with 25 μM CDDP without and with 100 μM TMPD, respectively (FIGS. 5c and 5d). The percentages also dramatically increased from 38% to 74% when cells were treated with 50 μM CDDP, without and with 100 μM TMPD, respectively (not shown). Consistent with cell cycle analysis (not shown here), it was observed that when CDDP was used as a treatment in combination with TMPD, the cells arrested in the early S phase and G1 phase of the cell cycle, whereas the cells tended to accumulate in the G2/M phase for lower doses of CDDP as a single agent. Interestingly, the dot plots indicated that more cells underwent DNA fragmentation when they were accumulated in the G1 phase. Although cells in all phases of the cell cycle will undergo apoptosis (non-specific), this will occur maximally for cells in the G1 phase. Thus, there is a significant increase (synergistic) in DNA fragmentation and apoptosis when TMPD is added in combination with CDDP, in correspondence with an earlier arrest of the cells in the S and G1 phases of the cell cycle.

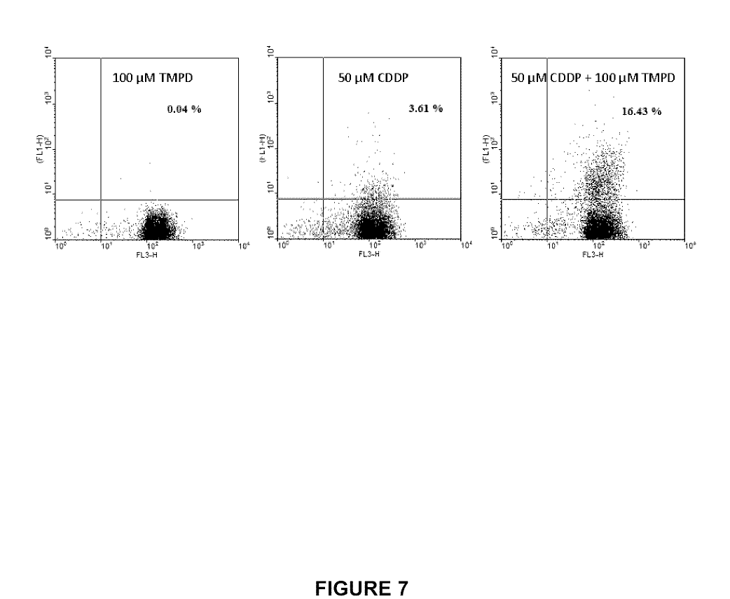

The data also shows that the combination treatment causes a significant increase of apoptotic cells in cisplatin-resistant HTB-161 cell line. As shown in FIG. 7, compared with the treatment of cisplatin alone, the combination treatment resulted in a 5-fold increase of DNA fragmentation, as indicated by the apoptosis assay, significantly increasing from 3.61% to 16.43% by combination with 100 μM TMPD, whereas the treatment with 100 μM TMPD in the vehicle alone showed only 0.04% fragmentation.

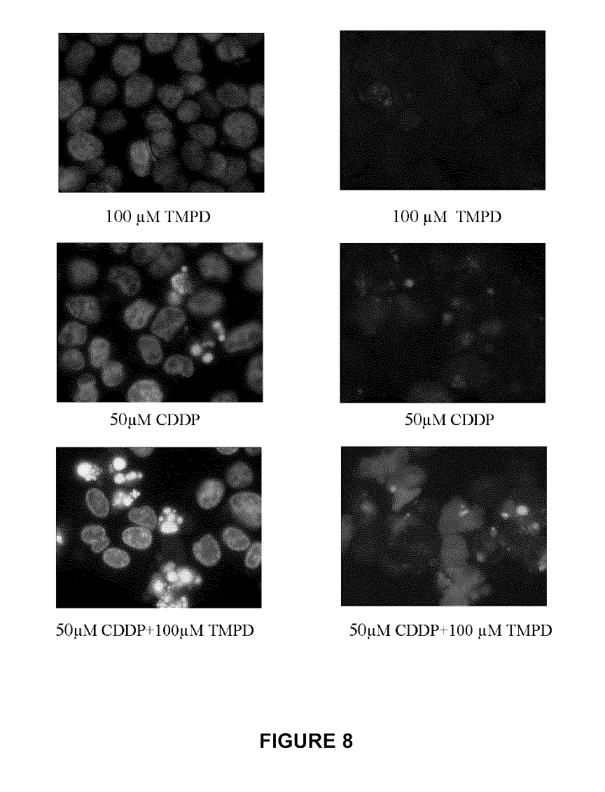

These results were further confirmed by fluorescence-based apoptosis assay (Invitrogen, Image-iT LIVE Green Caspase Detection Kit) after double staining for nuclear fragmentation/condensation by Hoechest 33342 staining (blue fluorescence) and caspase activation by FLICA reagent (green fluorescence) for PM2 and cisplatin in the cisplatin-resistant HTB-161 cells (FIG. 8). The images at the left are the cell nuclei detected by blue fluorescence of Hoechest 33342 staining, while those at the right are apoptotic cells detected by green fluorescence of FLICA reagent representing caspase activation. The presence of TMPD significantly increased the population of apoptotic cells, as can be clearly seen in the representative black & white images. The images clearly show the enhanced effect with the combined treatment.

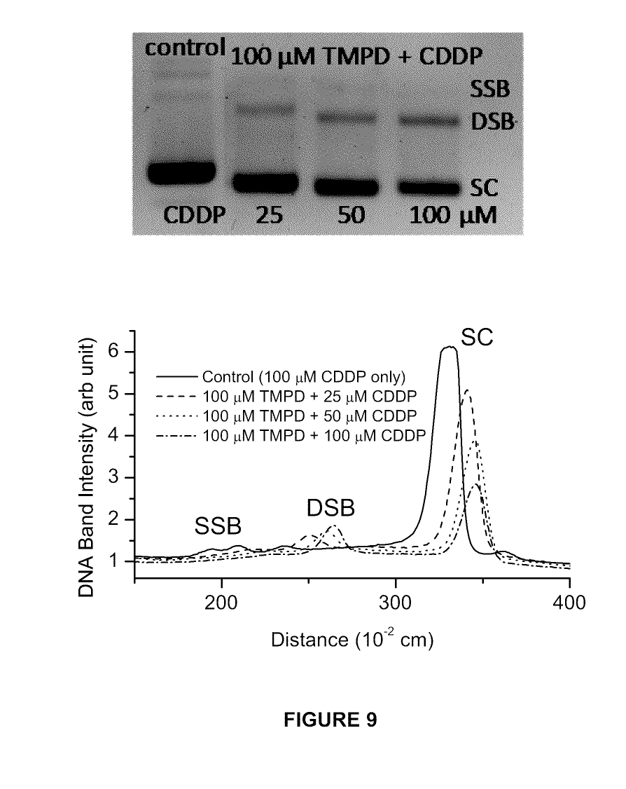

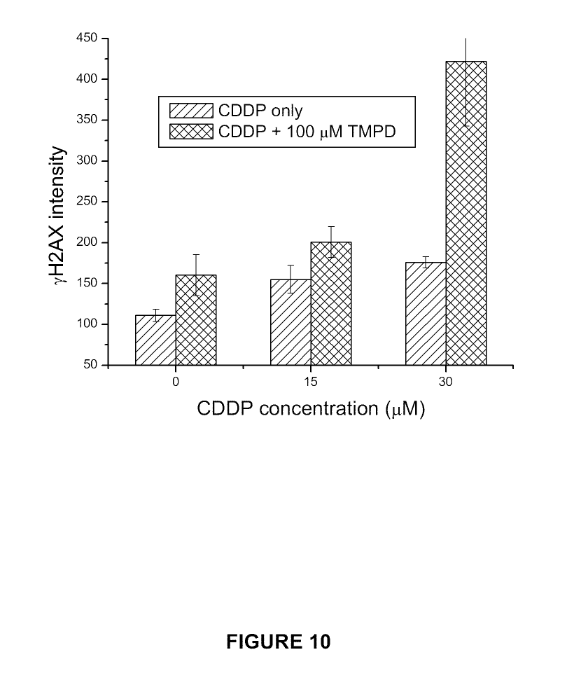

Referring to FIGS. 9 and 10, it was also investigated whether the DET reaction between cisplatin and one or two molecules of the molecular promoters would lead to an increase in DNA strand breaks. Double strand breaks are considered the most lethal as they cannot be effectively repaired by the cell. It is well known that monotreatment with cisplatin induces intrastrand cross links only, with no SSBs or DSBs, and this was confirmed even at very high cisplatin concentrations of up to 3.0 mM. In contrast, it is demonstrated that the combination of cisplatin with a biocompatible electron donor results in a significant amount of lethal DSBs in both plasmid DNA and genomic DNA in cervical cancer cells. Gel electrophoresis is a simple and reliable technique for quantitative measurements of plasmid DNA damage, characterizing single-strand breaks (SSBs), double-strand breaks (DBSs) and crosslinks (CLs).

The upper image in FIG. 9 shows agarose gel electrophoresis images of plasmid DNA in control, and 100 μM TMPD combined with 25, 50 or 100 μM cisplatin. The lower image shows densitograms of the gel image. As can be seen from FIG. 9, TMPD significantly increased the yield of double-strand breaks (DSBs) in plasmid DNA. FIG. 10 illustrates the results of γH2AX in HeLa cells treated with cisplatin alone and cisplatin plus 100 μM TMPD. The γH2AX intensity is proportional to the yield of DNA double-strand breaks (DSBs). The γH2AX DNA damage assay (Invitrogen) can been used to detect DSBs of genomic DNA in cancer cells. Briefly, DNA damaging radicals induce phosphorylation of histone variant H2AX (Ser139) forming DNA foci at the site of DNA DSBs. Phosphorylated H2AX aids in the recruitment of proteins responsible for DSB repair. These methods have been widely used for quantitative measurements of plasmid DNA damage and genomic DNA in cancer cells induced by radiation or anticancer agents. These results clearly illustrate an enhancement in cytotoxicity of cisplatin due to the combination treatment.

These results are particularly interesting, as DNA DSBs (unlike SSBs) are particularly difficult for the cell to repair and thus are potent inducers of cell death. These results strongly suggest that the combination treatment would effectively enhance DNA DSBs in vivo.

The in vitro results presented herein demonstrate that combination of ciplatin with a biocompatible electron donor, such as ICG or TMPD, enhances the potency of cisplatin, thereby reducing the required dose to kill tumor cells. Thus, the combination is expected to reduce the toxic side effects associated with cisplatin therapy in human patients. Incredibly, the combination treatment was also able to overcome cisplatin resistance in a well-established model of cisplatin-resistant cancer, thereby indicating that the combination treatment could likely broaden the use of cisplatin to other cancers that are typically resistant to cisplatin. Another advantage of this combination strategy is that the selected molecular promoters have very limited, if any, systemic toxicity themselves and dosages could be selected so as to minimize side effects from the promoter molecules.

### In Vivo Anticancer Effects of Combination Treatment

The in vivo therapeutic (e.g. anticancer) effects of a combination of a platinum-based antineoplastic agent and a biocompatible electron donor were investigated in the murine 4T1 breast cancer model, as outlined in Example 5. Two exemplary biocompatible electron donors, TMPD and ICG, were tested.

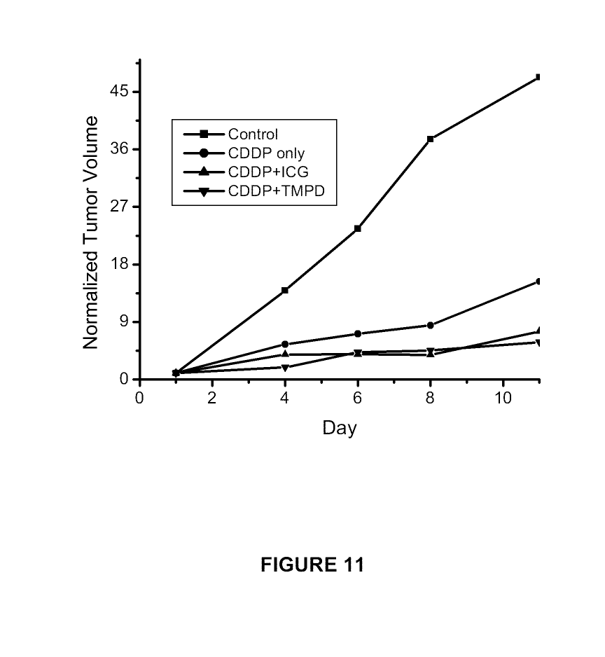

The combination of cisplatin with either ICG or TMPD significantly enhanced the suppression of tumor growth, compared with the treatment of cisplatin only in the tumor model, as can be seen from the tumor (volume) growth curves shown in FIG. 11. Tumor volumes were normalized to those just prior to drug treatment (i.e., at Day 1). Thus, the combination treatment with either cisplatin/ICG or cisplatin/TMPD significantly improved the antitumor effects of cisplatin in vivo. Given the difference in structure between the ICG and TMPD, it is predicted that these results can be extrapolated to various other compounds having electron-donating moieties capable of transferring electrons to cisplatin (or another platinum-based antineoplastic agent) in vivo to thereby synergistically enhance the chemotherapeutic effects of the anticancer agent.

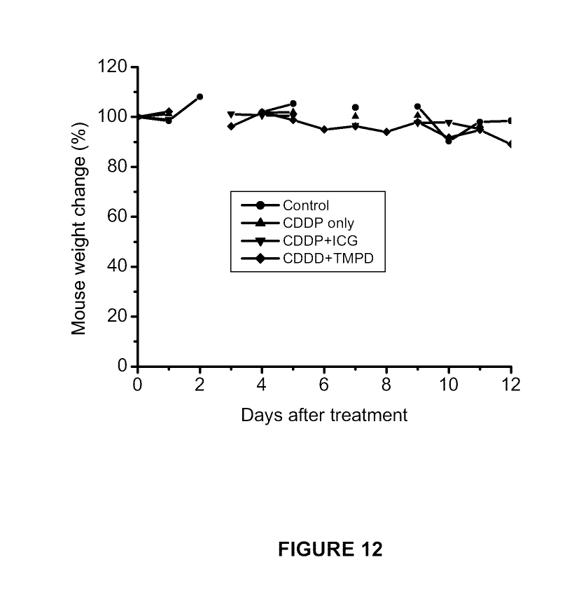

It should be noted that mice treated with the molecular promoters appeared healthy and displayed no signs of toxicity compared to controls or mice receiving cisplatin treatment only. As shown in FIG. 12, the weight changes of mice in all treatment groups were within 10%.

It is believed that the in vitro and in vivo results from the Examples provided herein can be extrapolated to other combinations, cancer cells, cancer models and human cancers beyond those exemplified. With the information provided herein, a rational approach can be used to identify other biocompatible electron donors besides those exemplified herein that can be used in combination with a platinum-based antineoplastic agent to enhance its anticancer effect. Various screening assays known to those skilled in the art can be used to assess the effect of a particular combination, as can the in vitro and in vivo experiments set forth in the Examples. Those combinations demonstrating synergy will be particularly preferred, as well as those that do not result in a net increase in toxic side effects compared to treatment with the antineoplastic agent alone. This rational approach to identifying effective compounds and combinations represents an efficient and economical alternative to random screening assays. Since the exemplary compounds tested herein are already in use in biological systems, the combinations disclosed herein represent synergistic combination treatments for cancer that can be easily moved forward into the clinical setting. Skilled professionals will readily be able to determine the effective amounts required for the combination therapy in vivo, e.g. so as to achieve the desired anticancer effect while reducing or maintaining levels of toxic side effects. Effective dosages may vary depending on the type and stage of cancer, the route of administration, the treatment regimen, among other factors. Studies can furthermore be conducted by skilled professionals in order to determine the optimal molecular ratio between the particular agents to be combined.

One advantage of the combination treatments disclosed herein is that the disclosed DET reaction mechanism is designed to be preferentially active at tumor cells due to the hypoxic microenvironment of tumors. In contrast to the environment of normal cells, where a molecular promoter will rapidly become oxidized and lose its electron-donating capability, thus the DET will not occur or its reaction efficiency will be significantly lowered in normal tissue. In cancer biology, the term “tumor environment” is often associated with tumor hypoxia. The presence of hypoxia in solid tumors has multiple consequences for tumor progression and treatment outcome, some of which are only recently being explored. It is known that hypoxia in solid tumors is not only a major problem for radiation therapy but also leads to resistance to many anticancer drugs. However hypoxia can be used as an advantage where a drug or prodrug is active preferentially in hypoxic cells, thereby reducing systemic toxicity. The unique presence of hypoxia in human tumors therefore provides an important target for selective cancer therapy.

Thus, one effort was the development of “bioreductive” drugs, which become activated under hypoxic conditions. Such an effort was hoped to open a new era in cancer research [13]. The leading bioreductive drug is tirapazamine (TPZ) [13,14]. Under hypoxia, TPZ is metabolized to a radical that produces DNA damage. In the presence of oxygen, the radical is converted (by oxidation) back to the parent compound. Some “bioreductive” drugs, such as TPZ, have shown enhancements in cytotoxicity when combined with cisplatin [14]. However, it is generally observed that there is no synergy between cisplatin and the bioreductive agents [13, 14]. Moreover, TPZ also induces toxic side effects [14]. TPZ is being evaluated in clinical trials. Despite promising results in some phase II trials, only a few randomized phase II or III clinical trials have been completed and have shown a limited improvement in tumor control or shown increased toxicity [14].

The biocompatible electron donors of the present disclosure display synergy when combined with cisplatin or other platinum-based anticancer agents since they are capable of donating one or more electrons to the antineoplastic agent to thereby enhance its anticancer activity. Preferably, the biocompatible electron donor is selected such that it is preferentially effective in donating one or more electrons to the platinum-based antineoplastic agent in the hypoxic tumour microenvironment.

### Definitions and Non-Limiting Embodiments

It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular forms “a”, “an”, and “the” include plural references unless indicated otherwise.

Terms of degree such as “substantially”, “about” and “approximately”, as used herein, mean a reasonable amount of deviation of the modified term such that the end result is not significantly changed. These terms of degree should be construed as including a deviation of at least ±5% of the modified term if this deviation would not negate the meaning of the word it modifies.

The term “subject” refers to a human or an animal to be treated, in particular, a mammal. Mammalian animals may include, for example, primate, cow, sheep, goat, horse, dog, cat, rabbit, rat, or mouse. The terms “subject” and “patient” are used interchangeably herein.

The term “cancer” (e.g. neoplastic disorder) as used herein refers to a disorder involving aberrant cell growth, proliferation or division (e.g. neoplasia). As neoplastic cells grow and divide they pass on their genetic mutations and proliferative characteristics to progeny cells. A “tumour” (e.g. neoplasm) is an accumulation of neoplastic cells. The methods and combinations disclosed herein may be used in the treatment of cancer, neoplastic cells, tumors and/or symptoms associated therewith.

Exemplary types of cancer that may be treated in accordance with the methods, uses and combinations of the present disclosure include, but are not limited to, testicular cancer, bladder cancer, cervical cancer, ovarian cancer, breast cancer, prostate cancer, head cancer, neck cancer, lung cancer (e.g. non small cell lung cancer), endometrial cancer, pancreatic cancer, Kaposi's sarcoma, adrenal cancer, leukemia, stomach cancer, colon cancer, rectal cancer, liver cancer, esophageal cancer, renal cancer, thyroid cancer, uterine cancer, skin cancer, oral cancer, brain cancer, liver cancer, gallbladder cancer. The cancer may, for example, include sarcoma, carcinoma, melanoma, lymphoma, myeloma, or germ cell tumours. In some embodiments, the cancer is testicular cancer, bladder cancer, cervical cancer, ovarian cancer, breast cancer, prostate cancer, head cancer, neck cancer, or lung cancer (e.g. non small cell lung cancer).

Any cancer that may be treated with a platinum-based antineoplastic agent may be treated in accordance with a method or combination of the present disclosure. Advantageously, cancers that are generally resistant or become resistant to treatment with a platinum-based antineoplastic agent may also benefit from treatment with a method, use or combination as disclosed herein, including but not limited to breast and prostate cancer.

An “anti-neoplastic agent” (or anti-cancer agent) refers to a therapeutic agent that directly or indirectly kills cancer cells, for example, by triggering apoptosis, or directly or indirectly prevents, stops or reduces the proliferation of cancer cells. In some cases, an “anti-antineoplastic agent” may include more than one therapeutic agent.

The terms “treat,” “treating” and “treatment” include the eradication, removal, amelioration, modification, reduction, management or control of a tumor, tumor cells or cancer, or the minimization, prevention or delay of metastasis.

The term “metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

The term “therapeutically effective amount” or “effective amount” is intended to mean that amount of a therapeutic agent (e.g. antineoplastic agent), or combination of agents (which may include a combination of a therapeutic and non-therapeutic agent), that will elicit a desired biological or medical response in a cell, tissue, growth, system, animal or patient, which result is generally sought by a researcher, veterinarian, doctor or other clinician or technician, including but not limited to, for example, reduction, prevention or elimination of cancer cells, a tumor, or cancer; reduced or inhibited cancer cell proliferation; increased or enhanced killing or apoptosis of cancer cells; or reduction or prevention of metastasis. In some cases, a desired biological or medical response may be amelioration, alleviation, lessening, or removing of one or more symptoms of cancer, or reduction in one or more toxic side effects associated with a particular cancer treatment.

When referring to the effective amount of a biocompatible electron donor to be administered in combination with an antineoplastic agent, the “effective amount” of the biocompatible electron donor may be an amount sufficient to produce a desired anticancer effect alone (if the compound itself has anticancer effects), or an amount sufficient to produce a desired anticancer effect of the antineoplastic agent. Similarly, when referring to a combination, the effective amount of the antineoplastic agent is an amount sufficient to provide a desired anticancer effect of the antineoplastic agent in the presence of the compound with which it is combined. An “anticancer effect” may include, but is not limited to, reduction, prevention or elimination of cancer cells, a tumor, or cancer; increased or enhanced killing or apoptosis of cancer cells; or reduction or prevention of metastasis.

In accordance with the present disclosure, a platinum-based antineoplastic agent is administered in combination with a biocompatible electron donor. The combination is such that the combined agents will at some point be co-present in the blood, or at the site of tumour cells or a tumour, although the agents need not be administered simultaneously in time or together in the same composition.

Synergistic combinations are particularly desirable. In some embodiments, the combination exhibits a synergistic anticancer effect. The terms “synergistic” and “synergy” imply that the effect of the combined agents is greater than the sum of the effects of the individual agents when administered alone.

By “inhibiting” or “reducing”, e.g. cancer cell proliferation, it is generally meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are either not treated or are not subjected to the methods and combinations of the present application.

By “reducing” a tumor it is generally meant to reduce the size of a tumour, as measured using methods known to those of ordinary skill in the art, by, for example, 10%, 20%, 30%, 40%, 50%, 60%, 70%, 80%, 90%, 95%, or 100%, when compared to tumor size before treatment or compared to tumors that are not subjected to the methods and combinations of the present application.

By “increased” or “enhanced” killing or apoptosis of cancer cells, it is generally meant an increase in the number of dead or apoptotic cells, as measured using methods known to those of ordinary skill in the art, by, for example, 10%, 20%, 30%, 40%, 50%, 60%, 70%, 80%, 90%, 95%, 100%, 200%, 300% or more, when compared to cells that are either not treated or are not subjected to the methods and combinations of the present application. An increase in cell killing or apoptosis could also be measured as a decrease in cell viability, as measured using a standard cell viability assay.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

As used herein, “platinum-based antineoplastic agent” generally refers to any platinum-containing drug that may be used in the treatment of cancer, including but not limited to, cisplatin, carboplatin, nedaplatin, oxaliplatin, satraplatin, triplatin tetranitrate, and analogues and derivatives thereof. In particular, platinum-based antineoplastic agents that benefit synergistically from combination with a biocompatible electron donor are contemplated herein.

In some embodiments, the “platinum-based antineoplastic agent” is cisplatin. In some embodiments, the “platinum-based antineoplastic agent” is carboplatin. In some embodiments, the “platinum-based antineoplastic agent” is nedaplatin. In some embodiments, the “platinum-based antineoplastic agent” is oxaliplatin. In some embodiments, the “platinum-based antineoplastic agent” is satraplatin. In some embodiments, the “platinum-based antineoplastic agent” is triplatin tetranitrate.

The terms “compound”, “molecule” and “agent” may be used interchangeably herein. Although platinum-based antineoplastic agents are technically referred to as metal complexes, they are referred to herein as compounds, molecules or agents.

An “analogue”, as used herein, generally includes molecules that have a similar function as a molecule to which it being compared, although the structure may differ. A “derivative”, as used herein, generally includes a molecule which, in one or multiple steps, could be converted back to the molecule of which it is a derivative. Derivatives include but are not limited to, for example, salt forms of molecules, pegylated molecules, and prodrug forms of molecules.

As used herein, “prodrug” generally refers to a compound that, upon in vivo administration, is metabolized or otherwise converted to the biologically, pharmaceutically or therapeutically active form of the compound. The prodrug approach is a chemical approach using reversible derivatives, which can be useful in optimizing the clinical application of a therapeutic agent. Prodrugs have been designed and developed, for example, to overcome pharmaceutical and pharmacokinetic barriers in clinical drug application, such as low oral drug absorption, lack of site specificity, chemical instability, toxicity, and poor patient acceptance. Conversion of a prodrug to the active form of the molecule can take place enzymatically or metabolically, or can take place by a number of additional mechanisms depending on, for example, on changes of pH, oxygen tension, temperature or salt concentration or by spontaneous decomposition of the drug or internal ring opening or cyclisation. For example, prodrugs can be produced such that they are converted to their active form in a hypoxic tumor environment. Additionally, prodrugs can be converted to active compounds by chemical or biochemical methods in an ex vivo environment.

As used herein, “biocompatible electron donor” generally refers to a molecule that is suitable for administration to a cell, tissue, patient (e.g. cancer patient) and that is capable of transferring one or more electrons to a platinum-based antineoplastic agent to thereby enhance its anticancer effect. The chemical interaction between the antineoplastic agent and the electron donor results in a synergy between the two compounds that enhances the cytotoxic effect of the antineoplastic agent.

In some embodiments, the biocompatible electron donor does not itself exerts any anticancer effects. In other embodiments, the biocompatible electron donor does exert anticancer effects. In still other embodiments, the biocompatible electron donor exerts therapeutic effects that are not anticancer effects, such as, for example, antibiotic effects or anti-inflammatory effects. Preferably, the biocompatible electron donor does contribute significant toxic effects to the combination, although it is to be understood that cancer therapy in general produces some toxic effects. Skilled persons will be readily able to titrate the dosages to minimize toxic effects while maximizing therapeutic benefit.

An “electron donor”, in its general sense, is a chemical entity that is capable of donating electrons to another compound. It is essentially a reducing agent that, by virtue of donating its electrons, is itself oxidized in the process. An electron donor may therefore also be referred to as a reducing agent. Certain conditions will enhance the reducing power of the electron donor, as will be understood by those of skill in the art. Many known reducing agents are not suitable for administration to patients however. Thus, this disclosure is concerned with biocompatible electron donors.

In some embodiments, the biocompatible electron donor is a small-molecule. A “small molecule”, as used herein, generally means a low molecular weight (e.g. less than 1000 Da, often less than 800 Da, often than 500 Da) organic compound. For example, in some embodiments, the biocompatible electron donor is a small molecule of less than about 1000 Da, less than about 800 Da, less than about 700 Da, less than about 600 Da, less than about 500 Da, less than about 400 Da, less than about 300 Da, less than about 200 Da, less than about 150 Da, or less than about 100 Da. In some embodiments, the biocompatible electron donor is a small molecule between about 100 Da and 1000 Da or between about 100 Da and 800 Da. In some embodiments, the biocompatible electron donor is a small molecule between about 100 Da and 200 Da, between about 200 Da and 300 Da, between about 300 Da and 400 Da, between about 400 Da and 500 Da, between about 500 Da and 600 Da, between about 600 Da and 700 Da, between about 700 Da and 800 Da, between about 800 Da and 900 Da, or between about 900 Da and 1000 Da. In some cases, a subunit of a polymer, or a small peptide, can be considered within the definition of a small molecule.

The biocompatible electron donor contains one or more electron-donating moieties. An electron-donating moiety may be present in the core structure of the molecule (e.g. a ring system which can act to stabilize a change), or in electron-donating substituents attached to the core structure, or a combination of the above.

In some embodiments, the biocompatible electron donor comprises one or more atoms having a lone electron pair. The lone electron pair may be associated with, for example, an O, N or S atom. In some embodiments, the atom having a lone pair may be a heteroatom in a heterocyclic or heteroaromatic ring. A heteroaromatic or heterocyclic ring may, for example, include single ring and multiple ring structures. In some embodiments, the ring may be integrated into the backbone structure of the molecule. Multiple-ring structures may include various combinations of aromatic (e.g. aryl), cyclic (e.g. cycloalkyl), heteroaromatic (e.g. heteroaryl) and heterocyclic (e.g. heterocycloalkyl) rings.

In some embodiments, the one or more atoms having a lone electron pair is present in an electron-donating substituent. An “electron donating” substituent refers to the ability of a substituent to donate electrons relative to that of hydrogen if the hydrogen atom occupied the same position in the molecule. This term is well understood by one skilled in the art and is discussed in Advanced Organic Chemistry, by J. March, John Wiley and Sons, New York, N.Y., pp. 16-18 (1985) and the discussion therein is incorporated herein by reference.

In some embodiments, the electron-donating substituent is coupled, either directly, or indirectly (e.g. within a few atoms), to a ring structure capable of stabilizing (e.g. assisting in stabilizing) a charge following donation of an electron. In some embodiments, the ring structure capable of stabilizing a charge following donation of an electron includes one or more aryl or heteroaryl moieties (aryl or heteroaryl moieties, in general, including but not limited to, 4-, 5- or 6-membered rings), which may be further coupled to other aromatic or non-aromatic moieties. The lone pair may be situated on an atom in an aromatic or non-aromatic moiety but preferably the charge can be stabilized through shifting of the charge around the molecule.

In some embodiments, the electron-donating substituent is −O, —OR, —OH, —SR, —SH, —NH₂, —NHR, or —NR₁R₂, —NHCOCH₃, —NHCOR, —OCH₃. In some embodiments, R, R₁ and R₂, which can be the same or different, are selected from the group consisting of substituted or unsubstituted alkyl, alkenyl, alkynyl, aryl, and aralkyl.

In some embodiments, the electron-donating group is NR¹R₂ and R₁ and R₂ are each alkyl, for example, methyl, ethyl, propyl or butyl. In some embodiments, the alkyl is methyl.

The following well-known chemical terms have the following general meanings, unless otherwise specified.

As used herein, the term “aryl” means a substituted or unsubstituted monovalent cyclic aromatic hydrocarbon moiety, for example, consisting of a mono-, bi- or tricyclic aromatic ring, including but not limited to those aryl groups in the molecules disclosed or exemplified herein.

The term “alkyl” denotes a saturated straight- or branched-chain group containing, e.g., from 1 to 10 carbon atoms, sometimes from 1 to 6 carbon atoms, sometimes from 1 to 4 carbon atoms, for example, methyl, ethyl, propyl, isopropyl, n-butyl, i-butyl, 2-butyl, t-butyl including but not limited to those alkyl groups in the molecules disclosed or exemplified herein.

The term “alkoxy” denotes a group wherein the alkyl residues are as defined above, and which is attached via an oxygen atom, e.g. methoxy and ethoxy.

The term “alkenyl” denotes a carbon chain of from 2 to 12, sometimes from 2 to 6, carbon atoms comprising a double bond in its chain. For example, C₂₋₆-alkenyl groups, include, e.g., ethenyl, propen-1-yl, propen-2-yl, buten-1-yl, buten-3-yl, penten-1-yl, penten-2-yl, penten-3-yl, penten-4-yl, hexen-1-yl, hexen-2-yl, hexen-3-yl, hexen-4-yl and hexen-5-yl.

The term “cyclic ring” or “cycloalkyl” denotes a monovalent or divalent saturated carbocyclic moiety consisting of a monocyclic ring containing, for example, 3 to 6 carbons. Cycloalkyl can optionally be substituted with one or more substituents.

The term “heterocyclic ring” or “heterocycloalkyl” means a monovalent saturated moiety, consisting of a ring having 4 to 7 atoms as ring members, sometimes 5 to 6 atoms as ring members, including one or more heteroatoms chosen from nitrogen, oxygen or sulfur, the rest of the ring members being carbon atoms. The heterocycloalkyl can optionally be substituted with one or more substituents, independently at each position. Examples of heterocyclic moieties include but are not limited to those in the molecules disclosed or exemplified herein.

A “heteroaryl” moiety is a mono- or polycyclic aromatic system, which is optionally substituted, containing at least one ring heteroatom selected from nitrogen, oxygen, and sulfur. Heteroaryl moieties may, for example, include aromatic rings having 5 or 6 ring atoms as ring members containing one or more heteroatoms selected from N, O, or S, the rest of the ring members being carbon atoms. Heteroaryl moieties can optionally be substituted, independently at each position. Examples of heteroaryl moieties include but are not limited to those in the molecules disclosed or exemplified herein.

The term “amine” may refer to an organic compound or functional group (i.e. amino) that contains a basic nitrogen atom with a lone electron pair, including, primary amine (NRH₂), secondary amine (NR₁R₂H), and tertiary amine (NR₁R₂R₂) where each R may be the same or different. Also, two R groups may denote members of a ring, e.g., where N is a heteroatom in a heterocyclic or heteroaryl ring.

The descriptions of compounds of the present application are limited by principles of chemical bonding known to those skilled in the art. Accordingly, where a group may be substituted by one or more substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

In some embodiments, the biocompatible electron donor is capable of donating two or more electrons, for example, 2, 3, 4, 5, 6, 7, 8, 9 or 10 electrons. In cases where an electron-donating compound has multiple electrons to donate, one molecule of the biocompatible electron donor may be capable of donating electrons to multiple molecules of the antineoplastic agent. It is believed that, where the antineoplastic agent receives two electrons, these may be received the same or different molecules of the biochemical electron donor depending on, for example, the particular donor molecule, the physiological conditions and the amount of donor present compared to the antineoplastic agent. Preferably, the biocompatible electron donor is preferentially capable of transferring one or more electrons to the antineoplastic agent in a tumour environment (e.g. under hypoxic conditions).

In some embodiments, the biocompatible electron donor is capable of donating two electrons to the antineoplastic agent, meaning that it has two electrons that could potentially be donated under suitable physiological conditions to a single molecule or two different molecules of the antineoplastic agent.

In some embodiments, the biocompatible electron donor has a redox potential between 0 and +1.0 V. By way of example, TMPD has a redox potential of +0.27 and ICG has a redox potential of +0.76 V.

In some embodiments, an electron-donating moiety may include a heteroatom having a lone pair, the heteroatom may be present in a ring having a substituent capable of enhancing the basicity of the heteroatom thereby increasing its properties as an electron-donating ligand.

In some embodiments, the biocompatible electron donor is an amine compound having at least two functional groups that contain a basic nitrogen atom and two methyl subgroups, in which the nitrogen atom features a lone electron pair and the effect of alkyl groups raises the energy of the lone pair of electrons. For example, the present inventor has found that such compounds are capable of easily donating electrons to activate cisplatin.

In some embodiments, the biocompatible electron donor is selected from the group consisting of: amine compounds; phenolic compounds; and quinones.

In some embodiments, the biocompatible electron donor is an amine compound that comprises at least one nitrogen atom having a lone electron pair. In some embodiments, the amine compound further comprises two alkyl substituents positioned on the molecule so as to increase the basicity of the nitrogen atom. The position will vary depending on the structure of the molecule.

In some embodiments, the biocompatible electron donor is N,N,N′,N′-tetramethyl-p-phenylene diamine (TMPD) or indocyanine green (ICG).

In one embodiment, the biocompatible electron donor is TMPD. In one embodiment, the biocompatible electron donor is ICG.

In some embodiments, biocompatible electron donor is a phenolic compound, such as a phenol or a polyphenol. In some embodiments, the biocompatible electron donor is a polyphenol. In some embodiments, the polyphenol is a flavanol (e.g. catechin). In some embodiments, the flavonol is epigallocatechin gallate (EGCG), epigallocatechin (EGC), epicatechin gallate (ECG) or epicatechin (EC). The flavonol may be from a natural source, for example, tea or fruit. In one embodiment, the flavonol is from green tea. In one embodiment, the flavonol is from grapes. The flavonol may, for example, be administered in a purified form or in an extract.

In some embodiments, the biocompatible electron donor is a quinone, such as a benzoquinone, naphthoquinone or anthraquinone.

Some exemplary biocompatible electron donors include, but are not limited to, e.g., indocyanine (ICG); N,N,N′,N′-tetramethyl-p-phenylene diamine (TMPD); 2,2,4-Trimethyl-1,3-pentanediol; p-(N,N-dimethylamino)phenol (DMAP); p-aminophenol; p-phenylenediamine; o-phenylenediamine; resorcinol; 4-methoxyphenol; Phenol; Dichlorophenolindophenol; cytochrome c; 1,4-benzoquinone; 1,4-naphthoquinone; or 9,10-anthraquinone.

In some embodiments, the biochemical electron donor has one or a combination of the following characteristics:

preferentially donates electrons to the platinum-based antineoplastic agent under hypoxic conditions, such as in a tumour microenvironment;

is capable of entering a cell, such as the nucleus of a cancer cell;

exhibits minimal toxic side effects in vivo.

The combination therapies disclosed herein include a combination of a platinum-based antineoplastic agent and a biocompatible electron donor. However, it will be understood that the combinations disclosed herein may be administered in further combination with one or more additional therapeutic agents. For instance, the additional therapeutic agent may include, e.g., an antibiotic, anti-inflammatory or another anticancer agent. Such additional anticancer agents may include, for example, classic chemotherapeutic agents, as well as molecular targeted therapeutic agents, biologic therapy agents, and radiotherapeutic agents. Anticancer agents used in further combination with the combined agents of present disclosure may include agents selected from any of the classes known to those of ordinary skill in the art, including, for example, alkylating agents, anti-metabolites, plant alkaloids and terpenoids (e.g., taxancs), topoisomerase inhibitors, anti-tumor antibiotics, hormonal therapies, molecular targeted agents, and the like. Generally such an anticancer agent is an alkylating agent, an antimetabolite, a vinca alkaloid, a taxane, a topoisomerase inhibitor, an anti-tumor antibiotic, a tyrosine kinase inhibitor, or an immunosuppressive macrolide. It will be understood that the additional agents selected should not significantly interfere with the combination therapy of the present disclosure so as to significantly reduce effectiveness of the combination therapy or enhance its toxic side effects.

The platinum-based antineoplastic agent and the biocompatible electron donor disclosed herein may be administered to a subject in need thereof in the form of a pharmaceutical composition, in one of various pharmaceutical dosage forms.

A “pharmaceutical composition” refers to a combination of ingredients that facilitates administration of one or more agents of interest (e.g. a therapeutic agent or a co-administrable agent) to an organism, e.g. a human or animal. A pharmaceutical composition generally comprises one or more agents of interest together with one or more pharmaceutically acceptable carriers or excipients. Many pharmaceutically-acceptable “carriers” or “excipients” are known in the art and these generally refers to a pharmaceutically-acceptable materials, compositions, or vehicles, including liquid or solid fillers, diluents, excipients, solvents, binders, or encapsulating materials. Each component in the composition must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams &amp; Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004). It will be understood that chemotherapy compositions will generally be associated with some level of toxicity and the clinician can weigh the risk/benefit ratio to determine the appropriate dosage and treatment regimen.

The combination therapies disclosed herein are typically administered to individuals who have been diagnosed with cancer. However, in some cases, the combination therapy may be administered to individuals who do not yet show clinical signs of cancer, but who are at risk of developing a neoplastic disorder. Toward this end, the present application also discloses methods for preventing or reducing the risk of developing a neoplastic disorder.

In some embodiments, the platinum-based antineoplastic agent and the biocompatible electron donor will be present in a single composition. However, due to the reactivity of the molecules, in some embodiments, the platinum-based antineoplastic agent and the biocompatible electron donor will be present in separate compositions. Where desired, the diluent or vehicle may be selected so as to protect the molecules, either individually or when combined together, from reacting until a desired timepoint or physical location is reached, such as, after administration or at a tumour site. For example, if an electron donating compound it sensitive to oxidation, it may be administered in a vehicle that prevents or delays oxidation. In one embodiment, an easily-oxidizable compound (such as TMPD) is administered in ethanol (e.g. 0.5%, 1%, 2%, 5%, 7%, 10%, 15%, 20% or 25%).

As used herein, the term “combination therapy” means that, at some point during the treatment, two or more agents of interest will be co-present in the blood or, in particular, at the site of a tumour or tumour cells. The two or more agents of interest are necessarily administered together at the same time or in the same composition. The platinum-based antineoplastic agent and the biocompatible electron donor may, for example, be administered simultaneously or concurrently (e.g. at substantially the same time) or separately (e.g. staggered times), for example, sequentially. In certain examples, the individual components of the combination may be administered separately, at different times during the course of therapy, or concurrently, in divided or single combination forms.

In some embodiments, the platinum-based antineoplastic agent and the biocompatible electron donor are administered simultaneously (e.g. together at the same time, or within 30 seconds of each other). The two agents may be administered simultaneously in a single composition or in separate compositions.

In some embodiments, the platinum-based antineoplastic agent and the biocompatible electron donor are administered in separate compositions.

In some embodiments, the platinum-based antineoplastic agent and the biocompatible electron donor are administered in sequentially. In some embodiments, the platinum-based antineoplastic agent and the biocompatible electron donor are administered sequentially, e.g. within 1 minute, 2 minutes, 5 minutes, 10 minutes, 20 minutes, 30 minutes, 40 minutes, 50 minutes, 1 hour, 1.5 hours, or 2 hours of each other.

In some embodiments, the biocompatible electron donor is administered before the platinum-based antineoplastic agent. In some embodiments, the biocompatible electron donor is administered following the platinum-based antineoplastic agent.

The pharmaceutical compositions disclosed herein may be formulated in any suitable dosage form, including single-unit and multiple-unit dosage forms. Exemplary dosage forms include, for example, a liquid, a solution, a suspension, an emulsion, a concentrate, a powder, a paste, a gel, a gum, a drop, a tablet, a capsule or a microcapsule. In some embodiments, the dosage form is a liquid. In some embodiments, the liquid is a solution, a suspension, or an emulsion.

The pharmaceutical composition as disclosed herein may comprise a platinum-based antineoplastic agent, a biocompatible electron donor, or both. In some embodiments, the composition comprises a biocompatible electron donor. In some embodiments, the composition comprises a biocompatible electron donor and a platinum-based antineoplastic agent. The composition may comprise one or more ingredients to stabilize (e.g. prevent or reduce the reactivity of) the biocompatible electron donor. Such ingredients will be known to those of skill in the part of formulations and will be dependent, at least in part, on the particular biocompatible electron donor. In general, biocompatible ingredients and reagents that prevent or slow oxidation of the biocompatible electron donor could be selected.

The compounds and compositions disclosed herein may be administered by any suitable route of administration. For example, the biocompatible electron donor and the platinum-based antineoplastic agent may, independently or together, be administered locally (e.g. into a tumor), regionally (e.g. into a body cavity) or systemically (e.g. into a blood vessel, such as a vein or artery). The biocompatible electron donor and platinum-based antineoplastic agent may be administered by the same route of administration or by different routes of administration. In some embodiments, the biocompatible electron donor and platinum-based antineoplastic agent are administered by the same route of administration. In some embodiments, the biocompatible electron donor and platinum-based antineoplastic agent are administered by different routes of administration.

In some embodiments, the pharmaceutical composition (comprising a platinum-based antineoplastic agent, a biocompatible electron donor, or both) is formulated for enteral administration, topical administration, parenteral administration, or nasal administration. Enteral administration may comprise, for example, oral administration.

In some embodiments, the biocompatible electron donor and the platinum-based antineoplastic agent are administered parenterally. Parenteral administration may comprise, for example, intravenous, intraarterial, intracerebral, intraperitoneal, intramuscular, subcutaneous, intracardiac, or intraosseous administration. In some embodiments, the parenteral administration is intravenous administration, e.g. injection or infusion. In some embodiments, the parenteral administration is intraarterial administration. In some embodiments, the parenteral administration is intraperitoneal administration.

In some embodiments, the parenteral administration is systemic or regional.

In some embodiments, the parenteral administration is systemic. In some embodiments, the biocompatible electron donor and platinum-based antineoplastic agent are administered intravenously in separate compositions.

The biocompatible electron donor and the platinum-based antineoplastic agent may be administered according to any treatment regimen deemed appropriate by the skilled worker (e.g. clinician). The biocompatible electron donor may be administered according to a similar or different regimen, as deemed appropriate by a skilled worker.

The dosage requirements of the compounds and pharmaceutical compositions disclosed herein will vary with the particular combinations employed, the route of administration and the particular cancer and cancer patient being treated. Treatment will generally be initiated with small dosages less than the optimum dose of the compound. Thereafter, the dosage is increased until the optimum effect under the circumstances is reached. In general, the compounds and compositions according to the present invention are most administered at a concentration that will generally afford effective results without causing any harmful or deleterious side effects. As with any chemotherapy, a certain degree of toxic side effects may be considered acceptable.

In some embodiments, the platinum-based antineoplastic agent (e.g. cisplatin) is administered parenterally (e.g. infused) for an administration period of about 30 minutes to about 2 hours per treatment session, e.g. at a rate of about 1 mg/min. In some embodiments, the antineoplastic agent is dissolved in a saline solution.

In general, a sufficient amount of the biocompatible electron donor should be employed to effectively react with the particular dose of antineoplastic agent employed.

In some cases, it is desirable to administer an excess of the biocompatible electron donor. For instance, since the biocompatible electron donor will generally be selected such that it is significantly less toxic than the antineoplastic agent, it may be administered in an excess dose. Also, since the reaction efficiency will generally be less than 100% (where not every molecule of electron donor will react with a molecule of the antineoplastic agent), it may also be desirable to administer an excess of the electron donor. In some embodiments, the excess is 1.5 times, 2 times, 2.5 times, 3 times, 5 times, or 10 times, the molar concentration of the platinum-based antineoplastic agent. For example, from the examples, a dose of ICG/TMPD that corresponds to doubling the concentration of cisplatin in μM, i.e., corresponds to the (weight) dose of 2×(775/300)=5.1× for ICG and 2×(164/300)=1.1× for TMPD, where x is the dose of cisplatin in mg/kg.

In some embodiments, for example, where a particularly effective electron donor is used, or one with many electrons to donate, the platinum-based antineoplastic agent may be administered in excess of the biocompatible electron donor.

Generally, a dose of the biocompatible electron donor will be selected such that the electron donor does not contribute significant toxic effects to the combination.

In one aspect, the present disclosure provides a method for treating cancer comprising administering to a subject in need thereof a therapeutically effective amount of a platinum-based antineoplastic agent and a biocompatible electron donor. In preferred embodiments, the biocompatible electron donor is capable of transferring one or more electrons to the platinum-based antineoplastic agent to thereby enhance its anticancer effect. Also provided are compounds, compositions, and dosage forms related to the combination therapy.

In preferred embodiments, the combinations of the present disclosure have a net anticancer effect that is greater than the anticancer effect of the individual components of the combination when administered alone. Preferably, the anticancer effect is increased without a concomitant increased toxic effect Thus, in another aspect, there is provided a synergistic combination comprising a platinum-based antineoplastic agent and a biocompatible electron donor for use in the treatment of cancer. Also provided is a synergistic combination comprising a platinum-based antineoplastic agent and a biocompatible electron donor for use in the manufacture of a medicament for the treatment of cancer. The combination is administered in a therapeutically effective amount. The features of the combination are as described in any of the embodiments disclosed herein above.

In another aspect, the present disclosure provides a use of a synergistic combination of a platinum-based antineoplastic agent and a biocompatible electron donor in the treatment of cancer. Also provided is a use of synergistic combination of a platinum-based antineoplastic agent and a biocompatible electron donor in the manufacture of a medicament for the treatment of cancer in a patient. In a accordance with the use, the combination is for administration in a therapeutically effective amount. The features of the use are as described in any of the embodiments disclosed herein above.

In another aspect, there are provided kits and commercial packages related to the combination therapy.

In some embodiments, a kit or commercial package is provided comprising a biocompatible electron donor and a platinum-based antineoplastic agent, together with instructions for carrying out a combination therapy for the treatment of a cancer.

In some embodiments, a kit or commercial package is provided comprising a pharmaceutical composition comprising a biocompatible electron donor and a platinum-based antineoplastic agent, together with instructions for carrying out a combination therapy for the treatment of a cancer.

In some embodiments, a kit or commercial package is provided comprising a first composition comprising a biocompatible electron donor; and a second composition comprising a platinum-based antineoplastic agent, together with instructions for carrying out a combination therapy for the treatment of a cancer.

In some embodiments of the methods, compositions, dosage forms, combinations, uses, kits and commercial packages, the platinum-based antineoplastic agent is cisplatin and the biocompatible electron donor is TPMD or ICG.

### EXAMPLES

The examples set forth below are intended to illustrate but not limit the scope of the disclosure.

### Example 1

### Synergy Between Cisplatin and ICG/TMPD in Treating Cisplatin-Sensitive Human Cervical Cancer (HeLa) Cells

### 1.1 Materials & Methods

1.1.1 Chemicals and Reagents

All compounds [cisplatin, indocyanine green (ICG, Scheme 1) and N,N,N′,N′-Tetramethyl-p-phenylene diamine (TMPD, Scheme 2), insulin and 3-(4,5-Dimethylthiazol-2-yl)-2,5-diphenyltetrazolium bromide (MTT)] were obtained from Sigma-Aldrich. MEM and fetal bovine serum (FBS), penicillin G and streptomycin were obtained from Hyclone Laboratories (UT, USA). The APO-BrdU™ TUNEL Assay Kit, RNase and propidium iodide were purchased from Invitrogen. Stock solutions of cisplatin and ICG were freshly prepared in ultrapure water, and stock solution of TMPD was prepared in pure ethanol, where the final concentration of ethanol was 0.2% when treated to cells.

1.1.2 Cell Culture

Cisplatin-sensitive human cervical cancer (HeLa) cells were purchased from the American Type Culture Collection (ATCC). Cells were cultivated with MEM (Hyclone) supplemented with 10% fetal bovine serum (Hyclone), 100 units/mL penicillin G and 100 μg/mL streptomycin (Hyclone). The cells were maintained at 37° C. in a humidified atmosphere containing 5% CO₂.

1.1.3 Cell Viability, Apoptosis and DNA Fragmentation Assays

1.1.3.1 Cell Survival Measurement by MTT

The synergetic effects of cisplatin and ICG/TMPD on cell viability were determined by the 3-[4,5-dimethylthiazol-2-yl]-2,5-diphenyl tetrazoliumbromide (MTT) assay. Cells were cultured in 96-well plates (5×10³ cells/well) for 24 h. The culture medium was replaced by new culture medium and incubated for 24 h with varying drug concentrations. After the drug treatment, cells were replaced with new medium containing 1.2 mM MTT (Sigma) and incubated for 4 h. The medium was then removed and the formazan crystals solubilized by 100 μL DMSO. The surviving fraction was determined by measuring the absorbance at 540 nm using a Multiskan Spectrum UV/Vis microplate reader (Thermo Scientific), which is directly proportional to the number of viable cells.

1.1.3.2 Cell Morphological Changes by Fluorescence Microscopy

Cells were cultured in black 96-well plates (˜5×10³ cells/well) for 24 h. The culture medium was replaced by new culture medium and incubated for 11 h with 0, 15, 30 and 50 μM CDDP (±50/100 μM ICG/TMPD). After the drug treatment, cells were gently washed once with PBS (Hyclone) and stained with Hoechst 33342 (Invitrogen) for 12 minutes. Cells were fixed with 1% paraformaldehyde in methanol and viewed under fluorescence using an Eclipse TS100 inverted microscope (Nikon Canada Inc.) and appropriate UV filters. Images were taken with a DS-Qi1Mc cooled digital camera (Nikon Canada Inc.).

1.1.3.4 DNA Fragmentation by Flow Cytometry

One late stage marker of apoptosis is the degradation of DNA into small fragments. The fragmentation of DNA in apoptotic cells leads to the disintegration of the nucleus, occurring between nucleosomes at ˜200-bp intervals. An enhancement in the amount of fragmented DNA can give indication to increases in apoptotic events, and thus to the viability of the drug combination.

Cells were cultured in 25 cm² flasks (˜8×10⁷ cells/flask) for 24 h. The culture medium was replaced by new medium and incubated for 48 h with varying drug concentrations. Both floating and adherent cells were harvested by trypsinization and fixed using 1% paraformaldehyde for 30 minutes. Cells were then permeabilized using 70% ice-cold ethanol for at least 24 h at −20° C. Samples were prepared for DNA fragmentation measurements using the APO-BrdU DNA Fragmentation Assay (Invitrogen). To label the DNA, ethanol was removed and cells were resuspended in 50 μL of TUNEL reaction buffer containing terminal deoxynucleotidyl transferase and BrdUTP for 1 h at 37° C., shaking every 15 min. After overnight room temperature incubation, the cells were then incubated with Alexa Fluor 488-conjugated BrdUTP-antibody for 30 min at room temperature. Cells were also stained with propidium iodide (5 μg/mL) and incubated with RNase A for 30 min at room temperature to determine the total cellular DNA content. Samples were then analyzed by a Becton-Dickinson FACSVantage SE flow cytometer in pulse processing mode (10 000 cells per sample). Computational analyses were performed using FCS Express software (De Novo Software, Los Angeles, Calif.) to obtain FL1-H versus FL2-A dot plots (log versus linear, respectively), gating on front scatter (FSC) versus side scatter (SSC) to eliminate debris, as well as FL2-A versus FL2-W to eliminate doublets. Experiments were performed independently at least 3 times

### 1.2 Results

1.2.1 Results of Experiment 1: Test on the Toxicity of ICG/TMPD

To evaluate the synergistic effects of ICG and TMPD in combination with CDDP on cell survival, the standard MTT assay was utilized. First, the cytotoxicity of ICG and TMPD alone was studied. Human cervical cancer (HeLa) cells were treated with various ICG or TMPD concentrations of 0-200 μM. The results are shown in FIGS. 1 and 2. It is clearly seen that ICG exhibits no toxicity even at very high concentration of 200 μM, and TMPD shows very mild toxicity up to the concentration f 100 μM. Thus, ICG and TMPD concentrations of no more than 100 μM were selected to avoid inducing additional toxicity in the combination therapy with cisplatin.

1.2.2 Results of Experiment 2: Synergy Between Cisplatin and ICG

FIG. 3a shows cell survival rates of HeLa cells after treatments with the combination of 15 μM cisplatin with various concentrations of ICG, indicating that ˜100 μM ICG is the highest concentration to achieve a maximum cytotoxicity. FIG. 3b shows the results for various concentrations (1-50 μM) of cisplatin (CDDP) alone, and the combination of 100 μM ICG with various concentrations of cisplatin. These results clearly demonstrate the synergetic effect of ICG in combination with cisplatin, which can significantly reduce the CDDP dose required to kill cancer cells effectively.

1.2.3 Results of Experiment 3: Synergy Between Cisplatin and TMPD

A similar synergetic effect of TMPD in combination with cisplatin is shown in FIG. 4, which plots cell survival rates of HeLa cells after treatments with the combination of various concentrations (0-50) μM of CDDP with and without 100 μM TMPD for 24 h. As shown in FIG. 2, 100 μM of TMPD provides a maximum dose having minimal cell-killing effects as a single agent. Treatment with 100 μM TMPD alone only slightly decreased the cell viability compared to the untreated cells. FIG. 4 indicates that a 24 h treatment with CDDP decreased the cell survival rates in a dose-dependent manner. Strikingly, the addition of 100 μM TMPD to CDDP greatly enhanced the effects of CDDP in a synergistic manner.

1.2.4 Results of Experiment 4: DNA Fragmentation Measurements

DNA fragmentation was measured by using the APO-BrdU TUNEL assay. The cell cycle distribution can also be detected simultaneously from the ordinate axis of the generated dot plot (with the BrdU-content on the abscissa), where the events with fluorescence intensity above the background indicate the cells with fragmented DNA (within upper box). The dot plots can also indicate the density of the events (number of events per pixel) by the colour, where blue indicates an area with high density of events and red indicates a low density.

HeLa cells were treated with 0, 10, 25 and 50 μM CDDP with and without 100 μM TMPD for 48 h. As shown in FIG. 5a, only 1% of untreated cells exhibited DNA fragmentation, which can be attributed to cells naturally undergoing cell death. Most of the events were in the G1 phase of the cell cycle, characteristic of a regular cell cycle distribution. FIG. 5b shows that 2% of cells treated with 100 μM TMPD alone had DNA fragmentation. When cells were treated with 10 μM CDDP+100 μM TMPD, the percentages increased from 9% to 18% compared to cells treated with only 10 μM CDDP (not shown here). Remarkably, the percentages increased from 6% to 69% when cells were treated with 25 μM CDDP without and with 100 μM TMPD, respectively (FIGS. 5c and 5d). The percentages also dramatically increased from 38% to 74% when cells were treated with 50 μM CDDP, without and with 100 μM TMPD, respectively (not shown here). Consistent with our cell cycle analysis (not shown here), it was observed that when CDDP was used as a treatment in combination with TMPD, the cells arrested in the early S phase and G1 phase of the cell cycle, whereas the cells tended to accumulate in the G2/M phase for lower doses of CDDP as a single agent. Interestingly, the dot plots indicated that more cells underwent DNA fragmentation when they were accumulated in the G1 phase. Although cells in all phases of the cell cycle will undergo apoptosis (non-specific), this will occur maximally for cells in the G1 phase. Thus, there is a significant increase (synergistic) in DNA fragmentation and apoptosis when TMPD is added in combination with CDDP, in correspondence with an earlier arrest of the cells in the S and G1 phases of the cell cycle.

1.2.5 Results of Experiment 5: Electron-Transfer Reaction of Cisplatin and ICG/TMPD

We have also demonstrated the electron-transfer reaction between cisplatin and ICG/TMPD by static and transient absorption spectroscopic measurements (no figures shown).

### 1.3 Discussion

Using the fast and convenient MTT assay, the percentages of cells which survived the drug treatments relative to the untreated cells were evaluated. These results suggest that although ICG/TMPD alone had minimal detrimental effects to the cells, the cell-killing effects were significantly increased when the combination of ICG/TMPD+CDDP was used. This indicates that the effects are synergistic since the effects of the combination were greater than the sum of the single-agent effects, thus supporting this combination chemotherapeutic regimen as a viable one.

The cytotoxicity enhancements of the combination treatments were also correlated with the increase in the amount of DNA fragmentation exhibited by the treated cells. Although ICG/TMPD, as a single agent, had minimal effects on DNA fragmentation of HeLa cells, it was shown that the addition of this compound to CDDP greatly increased the number of cells exhibiting DNA fragmentation. Furthermore, this synergistic enhancement also corresponded with earlier cell cycle arrests in the S and G1 phases, as compared to G2/M arrest for cells treated with only CDDP. This provides further evidence as to why and how these compounds are able to enhance the efficacy of CDDP on a cellular level.

We have also provided evidence of the strong electron-transfer reaction between ICG/TMPD and CDDP, consistent with the finding that CDDP is a very effective electron acceptor and the fact that ICG/TMPD is a well-known biological electron donor. The results have demonstrated how these compounds are able to enhance the efficacy of CDDP on a molecular level.

In summary, this example demonstrates that the dose of cisplatin (CDDP) required for cancer cell killing can be significantly reduced when administered in combination with a biocompatible electron donor, such as ICG/TMPD. Thus, this combination is expected to reduce the toxic side effects associated with the heavy-metal Pt-containing cisplatin in human cancer therapy.

### Example 2

### Combination of Cisplatin and TMPD Overcomes Drug Resistance in Cisplatin-Resistant Human Ovarian Carcinoma Cells

### 2.1 Materials & Methods

2.1.1 Chemicals and Reagents

All chemicals and reagents used in this study are the same as those listed in Section 1.1.1. In addition, the Image-IT™ LIVE Green Caspase-3 and -7 Detection Kit was purchased from Invitrogen.

2.1.2 Cell Culture

Cisplatin-resistant human ovarian cancer NIH-OVCAR-3 (HTB-161) cell line and RPMI 1640 cell-culture medium were obtained from American Type Culture Collection (ATCC). HTB-161 cells were cultured in RPMI 1640 supplemented with 20% fetal bovine serum, 0.01 mg/ml bovine insulin, 100 μg/ml streptomycin, and 100 units/ml penicillin. The cells were maintained at 37° C. in a humidified atmosphere containing 5% CO₂.

2.1.3 Cell Viability, Apoptosis and DNA Fragmentation Assay

2.1.3.1 Cell Viability

The effects of cisplatin and TMPD on cell viability were determined by 3-[4,5-dimethylthiazol-2-yl]-2,5-diphenyl tetrazoliumbromide (MTT) assay. Briefly, HTB-161 cells were plated at a density of 7×10³ cells/well in 96-well plates. Following overnight incubation, cells were treated with different concentrations of cisplatin and TMPD for 24 h. After incubation for specified times at 37° C. in a humidified incubator, 20 μL of MTT (5 mg/mL in PBS) were added to each well, and cells were incubated for a further 4 h. After removal of the medium, 100 μL DMSO was added to each well. The absorbance was recorded on a microplate reader at the wavelength of 540 nm. The effects of cisplatin and TMPD on growth inhibition were assessed as percent cell viability where 0.2% ethanol-treated cells were taken as 100% viable.

2.1.3.2 Detection of Apoptosis by Fluorescence Microscopy

The Image-IT™ LIVE Green Caspase-3 and -7 Detection Kit (Invitrogen) was used for the detection of active caspases and apoptotic bodies following the vendor's protocol. This kit provides a fluorescent inhibitor of caspases (FLICA) specific for caspase-3 and -7, the cells show green fluorescence for active caspase-3 and -7. The kit also includes Hoechest 33342 and propidium iodide (PI) stains, which allow the evaluation of nuclear morphology and plasma membrane integrity. HTB-161 cells were grown to 60% confluence and then treated with cisplatin and 100 μM TMPD for 10 h. The fluorescence was detected with a Nikon Eclipse TS100 microscope. Images were captured with an attached camera.

2.1.3.3 Quantification of DNA Fragmentation and Apoptosis by Flow Cytometry

The method based on terminal deoxyribonucleotidyl transferase (TdT)-mediated dUTP nick-end labelling was used to determine the cell apoptosis. A landmark of cellular self-destruction by apoptosis is the activation of nuclei that eventually degrade the nuclear DNA into fragments. Detection of these DNA fragments is relatively straightforward for identifying apoptotic cells. DNA fragmentation was measured by using the APO-BrdU TUNEL assay. HTB-161 cells were grown at a density of 60% confluence in T-25 flasks and were treated with cisplatin and TMPD for 24 h. After trypsinized, the cells were washed with PBS, were processed for labeling with the deoxythymidine analog 5-bromo-2′-deoxyuridine 5′-triphosphate (BrdUTP), and were incubated with an Alexa Fluor 488 dye-labeled anti-BrdU antibody and propidium iodide by the use of an APO-BrdU™ TUNEL Assay Kit (Invitrogen) as per manufacturer's protocol. The labeled cells were analyzed by flow cytometry.

2.1.3.4 Cell Cycle Analysis

HTB-161 cells were plated at a density of 2×10⁵ per well on six-well plates. After overnight incubation, cells were treated with cisplatin and TMPD for 24 h. The cells were trypsinized, washed twice with chilled PBS, and centrifuged. The cell pellet was resuspended in 70% ethanol at −20° C. overnight. The cells were centrifuged at 1,000 rpm for 5 min; the pellet was washed twice with chilled PBS, suspended in 500 μL PBS, and incubated with RNase (20 μg/mL final concentration) at 25° C. for 30 min and with propidium iodide (50 μg/mL final concentration) for an additional 30 minutes. The cells were analyzed by flow cytometry. Flow cytometry was done with a FACS vantage SE (Becton Dickinson). A minimum of 10,000 cells per sample were counted and the DNA histograms were further analyzed by using FCS Express software for cell cycle analysis.

### 2.2 Results

2.2.1 Results of Experiment 1: TMPD Makes Cisplatin-Resistant HTB-161 Cells Sensitive to Cisplatin

We investigated whether cisplatin in combination with TMPD modulates the tumor resistance to cisplatin. The experiment was conducted with ovarian cancer cell line, HTB-161, which is cisplatin-resistant. We compared the effects of cisplatin, TMPD and the combination of two compounds on cell viability. As shown in FIG. 6, HTB-161 cells still survive from the treatment of extremely high doses of cisplatin, showing strong resistance to the drug, while the combination treatment dramatically decreases the cell viability. The cell viability was about 40% when treated with cisplatin alone at an extremely high concentration of 250 μM, whereas all cells (100%) were strikingly killed when the treatment of cisplatin was combined with 100 μM TMPD. Note that the treatment with 100 μM TMPD alone only showed a slight decrease of the cell viability. Thus, the combination treatment showed a strong synergetic effect in HTB-161 cells. Most remarkably, the results in FIG. 6 indicate that the cisplatin resistance of the cells can be completely removed by the combination with TMPD.

2.2.2 Results of Experiment 2: TMPD Enhances Cisplatin-Induced DNA Fragmentation and Apoptosis in HTB-161 Cell Line

The APO-BrdU TUNEL assay was used to determine DNA fragmentation and cell apoptosis. As shown in FIG. 7, the treatment with TMPD alone at the concentration of 100 μM caused few BrdU positive cells (0.04%), which was similar to the treatment of the vehicle. In striking contrast, the combination treatment resulted in a significantly increase of BrdU positive cells: TMPD increases the DNA fragmentation of HTB-161 cells from 3.61% by treatment with 50 μM cisplatin alone to 16.43% by combination of 50 μM cisplatin with 100 μM TMPD. These data showed that the combination treatment causes a significant increase of apoptotic cells in cisplatin-resistant HTB-161 cell line.

2.2.3 Results of Experiment 3: TMPD Enhances Cisplatin-Induced Apoptosis and Activation of Caspases in HTB-161Cells

To test whether cisplatin and TMPD-mediated decrease in cell growth is due to induction of apoptosis, we use the Image-IT™ LIVE Green Caspase-3 and -7 Detection Kit. The results in FIG. 8 shows a significant induction of apoptosis when the cells were treated with 50 μM cisplatin plus 100 μM TMPD for 10 h. The Hoechest 33342 staining (blue) exhibits that the combination treatment significantly increases the population of nuclear damaged cells, compared with the treatment of cisplatin alone (left, FIG. 8). FLICA reagent was used to detect the activation of caspase enzymes. The result showed that the combination treatment resulted in a significant activation of caspase-3 and -7, which was evident from the significant enhancement in green fluorescence (Right, FIG. 8) (Red fluorescence of propidium iodide was hardly detected in the cells, this is due to few necrotic cells when treated in a short time).

2.2.4 Results of Experiment 4: Cell Cycle Analysis

To assess the effect of combination of cisplatin with TMPD on the distribution of cells in the cell cycle, we have also performed DNA cell cycle analysis. Our observed results (no figure shown) show that the treatment of cisplatin alone showed a accumulation of cells in the G1 phase of the cell cycle, and the combination of cisplatin with 100 μM TMPD exhibited the cell cycle profiles which resembled those of cells treated with cisplatin alone. However, there was an apparent increase in the percentage of hypodiploid cells (in sub-G1) for the combination of cisplatin with TMPD, compared with cisplatin alone, indicating the increase of apoptotic cells.

### 2.3 Discussion

Resistance to cisplatin in ovarian cancer presents a major clinical problem. To circumvent the drug resistance remains a challenging issue. One of the practical ways to solve this problem in the clinic is to combine cisplatin with a synergistic molecular promoter, thus improving the therapeutic efficacy without increasing toxicity. To identify an effective promoter, however, will require a precise understanding of the molecular mechanism of cytotoxic activity of cisplatin [1-3].

In the first experiments, it was demonstrated that two exemplary molecular promoters, indocyanine green (ICG) and N,N,N′,N′-Tetramethyl-p-phenylene diamine (TMPD), identified through new mechanistic understandings of cisplatin on a molecular level [5,6], result in large enhancements in the cytotoxic effect of cisplatin in a synergistic manner.

In this example, it is demonstrated that the combination effect of TMPD can effectively make the cisplatin-resistant ovarian carcinoma cells sensitive to cisplatin and that the drug resistance can be completely removed. This drastic improvement is due to our finding of the dissociative-electron-transfer (DET) mechanism of cisplatin [5, 6]. According to the DET mechanism, cisplatin is an extremely reactive compound with electrons (a highly oxidizing agent). As cisplatin enters a cell, it may react with electron-donating components in proteins other than G bases in DNA and thus losses its targeting to DNA (causing DNA damage). Indeed, intracellular cisplatin inactivation by glutathione has also been proposed as a mechanism of cisplatin resistance [1-3, 15, 16]. Glutathione is the most abundant intracellular thiol and serves as a critical cellular antioxidant (electron donor). Total cellular glutathione content also is an important determinant of cisplatin resistance [15]; there has been evidence that the glutathione level is associated with the degree of cisplatin resistance in ovarian cancer cells [16]. The presence of strong electron-donating molecules such as ICG/TMPD inside the cell and their highly effective reaction with cisplatin can compete with or suppress the reaction of cisplatin with glutathione. The resultant Pt(NH₃)₂Cl. or Pt(NH₃)₂. radical can then lead to DNA strand breaks and cell death.

The present in vitro tests have demonstrated that the combination treatment of cisplatin with TMPD can successfully circumvent the drug resistance of cisplatin against cisplatin-resistant ovarian cancer cells. This strongly encourages for in vivo studies of the novel combination therapy of cisplatin and ICG/TMPD in animal cancer models.

### Example 3

### Combination of Cisplatin and TMPD Induces DNA Double-Strand Breaks

### 3.1 Materials & Methods

3.1.1 Chemicals and Reagents

Ultrapure water for life science with a resistivity of >18.2 MΩ/cm and TOC<1 ppm obtained freshly from a Barnstead Nanopure water system was used. Chemicals (isopropanol, dimethylsulfoxide and KNO₃) were obtained from Sigma-Aldrich. Plasmid DNA [pGEM 3Zf(−), 3197 kp] was extracted from Escherichia Coli JM 109 and purified using QIAprep Kit (Qiagen). The γH2AX DNA damage assay kit was purchased from Invitrogen.

3.1.2 Agarose Gel Electrophoresis & the γH2AX DNA Damage Assay

The gel electrophoresis was used for quantitative measurements of plasmid DNA damage, characterizing single-strand breaks (SSBs), double-strand breaks (DBSs) and crosslinks (CLs). We also used the γH2AX DNA damage assay (Invitrogen) to detect DSBs of genomic DNA in cancer cells. Briefly, DNA damaging radicals induce phosphorylation of histone variant H2AX (Ser139) forming DNA foci at the site of DNA DSBs. Phosphorylated H2AX aids in the recruitment of proteins responsible for DSB repair.

Quartz cells contained 3.0 μg of DNA in 200 μL of aqueous solutions of 100 μM cisplatin, (25, 50 and 100 μM) cisplatin combined with 100 TMPD. Aliquots equivalent to 96 ng DNA were removed from the solutions for gel electrophoresis. All aliquots were analyzed with a standard agarose gel electrophoresis method, namely, on 1% neutral TAE agarose gel in TAE running buffer. The gel was prestained with 1 μg/ml ethidium bromide. The image of the gel was taken on Fluor Chem imaging station (Alpha Innotech) and exhibits various DNA topological forms, including supercoiled (SC, undamaged DNA), open circular (C, SSB) and linear form (L, DSB).

### 3.2. Results

As shown in FIG. 9, the combination of cisplatin with TMPD induces DNA strand breaks, most importantly double-strand breaks (DSBs). It is well known that the single treatment of cisplatin alone induces no SSBs and DSBs but intrastrand cross links only [4-6]. The present inventor confirmed this even at very high cisplatin concentrations of up to 3.0 mM (no figure shown). In contrast, the results shown here clearly demonstrate that the combination of cisplatin with TMPD results in a significant amount of DSBs in both plasmid DNA (FIG. 9) and genomic DNA in cervical cancer cells (FIG. 10).

### 3.3. Discussion

The results shown in FIGS. 9 and 10 demonstrate that the combination treatment effectively results in DNA DSBs. This finding is remarkable, as the use of radiation or DNA-damaging agents usually results in a significant amount of SSBs but a very small fraction of DSBs of DNA. These results are particularly interesting, as DNA DSBs (unlike SSBs) are well-known to be difficult for the cell to repair and thus potent inducers of cell death. This is consistent with what is observed for the cytotoxicity and apoptosis of the combination therapy of cisplatin with ICG or TMPD, as shown in FIGS. 1-8, and is consistent with the proposed dissociative-electron-transfer (DET) mechanism for the combination of cisplatin with biocompatible electron donors, such as ICG/TMPD.

### Example 4

### Effects of ICG and TMPD In Vivo in Combination with Cisplatin in the 4T1 Mouse Breast Cancer Model in Female BALB/c Mice

### 4.1 Materials and Protocol

4.1.1 Study Groups

The four groups in the experiment were: (1) control article (25% EtOH/water); (2) Cisplatin 3 mg/kg in 0.3% saline; (3) cisplatin+ICG 15.6 mg/kg in 0.3% saline; and (4) cisplatin+TMPD 3.3 mg/kg in 25% EtOH/water, as shown in Table 1.

TABLE 1
Study Groups in Example 4

| | | | TA/CA* | | | |
|---|---|---|---|---|---|---|
| Group | | No. | Dose | Admin. | Volume | Schedule |
| # | Group Name | mice | (mg/kg) | Route | (uL/20 g) | (Days) |
| 1 | Control (vehicle) | 4 | N/A | i.p. | 200 | 1, 5, 9 |
| 2 | Cisplatin | 4 | 3 | i.p. | 200 | 1, 5, 9 |
| 3 | Cisplatin + ICG | 4 | 3/15.6 | i.p. | 200 | 1, 5, 9 |
| 4 | Cisplatin + TMPD | 4 | 3/3.3 | i.p. | 200 | 1, 5, 9 |

*TA: Test Article; CA: Control Article
**treatment to be initiated~Day 11 post inoculation

4.1.2 Cell Preparation (Tissue Culture):

Tumorigenic mouse breast cancer cell line 4T1 was obtained from ATCC (Cat# CRL-2539). Cells were started from a frozen vial of lab stock which were frozen down from ATCC original vial and kept in lab liquid nitrogen tanks. Cell cultures with passage #3 to #10 and a confluence of 80-90% were harvested for in vivo studies. 4T1 cells were grown in the medium provided by ATCC. Cells were subcultured once a week with split ratio 1:4 or 1:8 and expanded. The medium was renewed twice a week.

4.1.3. Cell Preparation (Harvesting for s.c. Inoculation):

Cells were rinsed briefly one time with PBS. Fresh Trypsin/EDTA solution (0.25% trypsin with EDTA 4Na) was added, and flask was laid horizontally to ensure the cells were covered by trypsin/EDTA, and then the extra trypsin/EDTA was aspirated. Cells were allowed the to sit at room temperature for a few minutes, cells were dislodged by rapping the side of the flask sharply with the palm of the hand. Cells were observed under an inverted microscope until cell layer was dispersed, fresh growth medium was added, cells were centrifuged at 1,000 rpm for 7 min and the supernatant was aspirated. Cells were resuspended in PBS, and 50 μl of cell suspension was mixed with trypan blue (1:1) and counted and assessed for viability on a haemocytometer. Cells were centrifuged the at 1,000 rpm for 7 min, and supernatant was aspirated. The cells were re-suspend with HBSS to appropriate concentration for inoculation. Injection volume was 50 μl per animal.

4.1.4 Tumour Cell Implantation (Solid Tumours)

On day 0, tumour cells were implanted subcutaneously into female BALB/c mice (age 6-8 weeks) in a volume of 504 using a 27-gauge needle. Treatment was initiated between 1 and 2 weeks of inoculation, when tumours were measurable.

4.1.5 Dose Administration

Mice were individually weighed and injected intraperitoneally according to body weight for an injection concentration as outlined in the study group table above. The injection volume was based on 5004 per 20 g mouse. The abdominal surface was wiped down with 70% isopropyl alcohol to clean the injection site.

4.1.6 Data Collection

Tumour growth was monitored by measuring tumour dimensions with calipers beginning on first day of treatment. Tumour length and width measurements were obtained daily. Tumour volumes were calculated according to the equation L×W2/2 with the length (mm) being the longer axis of the tumour. Animals were also weighed at the time of tumour measurement. Tumours were allowed to grow to a maximum of 1000 mm3 before termination.

4.1.7 Evaluation of Drug Induced Stress in the Mice

All animals were observed post administration, and at least once a day, more if deemed necessary, during the pre-treatment and treatment periods for mortality and morbidity. In particular, animals were monitored for signs of ill health such as body weight loss, change in appetite, behavioural changes such as altered gait, lethargy and gross manifestations of stress.

### 4.2 Results

Briefly, to study in vivo growth inhibitory efficiencies of the combination treatments of cisplatin with ICG or TMPD (two exemplary biocompatible electron donors), six to eight week old female nude BALB/c mice were injected subcutaneously with 1×10⁷ 4T1 cells in the right hind flank. Mice were monitored daily for tumor growth by visual inspection and palpitation. Drug treatments were initiated between 1 and 2 weeks of inoculation, when tumours were measurable. Mice were randomly divided into four groups of 4 mice, and were treated with three doses at Day 1, 5, 9 of control (saline/EtOH), cisplatin (3 mg/kg) only, cisplatin (3 mg/kg cisplatin) plus 15.6 mg/kg ICG, and cisplatin (3 mg/kg cisplatin) plus 3.3 mg/kg TMPD groups. PM1 or PM2 and CDDP were administered by separate intraperitoneal (IP) injections. Four mice were randomly allotted per treatment group. Tumors were measured with a caliper twice per week and mice monitored for adverse side effects of the drugs. At necropsy, tumor length and width were measured, and approximate tumor volumes were calculated according to the equation: tumor volume (mm³)=length×(width)²/2.

The efficacy of the combination therapy of cisplatin plus ICG or TMPD, with respect to the monotherapy of cisplatin, is demonstrated by the tumor (volume) growth curves of the various treatment groups, as shown in FIG. 11. The tumor volumes were normalized to those just prior to drug treatment (i.e., at Day 1). It can be seen from FIG. 11 that the combination of cisplatin with either ICG or TMPD significantly enhanced the suppression of tumor growth, compared with the treatment of cisplatin only, in the 4T1 breast cancer mouse model. Given the difference in structure between the ICG and TMPD, it is predicted that these results can be extrapolated to various other compounds having electron-donating moieties capable of transferring electrons to cisplatin (or another platinum-based antineoplastic agent) in vivo to thereby synergistically enhance the chemotherapeutic effects of the anticancer agent.

No signs of toxicity or ill health, nor significant change in weight, were noted in any of the animals treated.

### REFERENCES

1. M. F. Fuertes, C. Alonso and J. M. Perez, Biochemical Modulation of Cisplatin Mechanisms of Action: Enhancement of antitumor Activity and Circumvention of drug Resistance, Chem. Rev. 103, 645 (2003).
2. J. Reedijk, New clues for platinum antitumor chemistry: Kinetically controlled metal binding to DNA, PNAS 100, 3611(2003).
3. D. Wang and S. J. Lippard, Cellular Processing of Pt Anticancer Drugs, Nature Reviews: Drug Discovery 4, 307 (2005)
4. D. M. Reese, Anticancer drugs, Nature 378, 532 (1995).
5. Q.-B. Lu, Molecular Reaction Mechanisms of Combination Treatments of Low-Dose Cisplatin with Radiotherapy and Photodynamic Therapy, J. Med. Chem. 50, 2601 (2007).
6. Q.-B. Lu, S. Kalantari and C.-R. Wang, Electron Transfer Reaction Mechanism of Cisplatin with DNA at the Molecular Level, Molecular Pharmaceutics 4, 624 (2007).
7. Swanson K L, Prakash U B S, Stanson A W, Pulmonary arteriovenous fistulas: Mayo Clinic experience, 1982-1997, MAYO CLINIC PROCEEDINGS 74, 671-680 (1999).
8. Malicka J, Gryczynski I, Geddes C D, et al., Metal-enhanced emission from indocyanine green: a new approach to in vivo imaging, J. BIOMED. OPTICS 8, 472-478 (2003).
9. P. Jurtshuk, Jr., D. N. McQuitty and W. H. Riley IV, Current Microbiology 2, 349-354 (1979).
10. http://www.atcc.org/
11. Rogan A M, et al. Reversal of adriamycin resistance by verapamil in human ovarian cancer. Science 224, 994-996 (1984).
12. Hamilton T C, et al. Characterization of a xenograft model of human ovarian carcinoma which produces ascites and intraabdominal carcinomatosis in mice. Cancer Res. 44, 5286-5290 (1984).
13. J. M. Brown, The hypoxic cell: A target for selective cancer therapy—Eighteenth Bruce F. Cain Memorial Award lecture, Cancer Research 59, 5863-5870 (1999).
14. S. R. McKeown, R. L. Coweny, K. J. Williamsy, Bioreductive Drugs: from Concept to Clinic, Clinical Oncology 19, 427-442 (2007).
15. Rudin C M, Yang Z, Schumaker L M, Vander Weele D J. Inhibition of glutathione synthesis reverses Bcl-2-mediated cisplatin resistance. Cancer Res 2003; 63: 312-318.
16. Fracasso P M. Overcoming drug resistance in ovarian carcinoma. Curr Oncol Rep 2001; 3:19-26.

All references recited herein are incorporated by reference in their entirety.

The above-described embodiments are intended to be examples only. Alterations, modifications and variations can be effected to the particular embodiments by those of skill in the art without departing from the scope, which is defined solely by the claims appended hereto.

1. A method for the treatment of a cancer comprising:
administering to a subject in need thereof a therapeutically effective amount of a platinum-based antineoplastic agent and a biocompatible electron donor.

2. The method of claim 1, wherein the biocompatible electron donor is capable of transferring one or more electrons to the platinum-based antineoplastic agent to thereby synergistically enhance its anticancer effect.

3. The method of claim 1 or 2, wherein the biocompatible electron donor comprises one or more atoms having a lone electron pair selected from the group consisting of O, N or S.

4. The method of claim 1, 2 or 3, wherein the one or more atoms having a lone electron pair is present in a heteroaryl ring or a heterocyclic ring.

5. The method of claim 1, 2 or 3, wherein the one or more atoms having a lone electron pair is present in electron-donating substituent is −O, —OR, —OH, —SR, —SH, —NH₂, —NHR, or —NR₁R₂, —NHCOCH₃, —NHCOR, —OCH₃, wherein R, R₁ and R₂ can be the same or different, and are selected from the group consisting of substituted or unsubstituted alkyl, alkenyl, alkynyl, aryl, and aralkyl.

6. The method of claim 5, wherein the electron-donating substituent is —NH₂, —NHR, or —NR₁R₂.

7. The method of claim 5 or 6, wherein the electron-donating substituent is coupled to a structure that is capable of stabilizing a charge following donation of an electron.

8. The method of claim 5, 6 or 7, wherein the electron-donating group is −NR₁R₂ and wherein R₁ and R₂ are each methyl.

9. The method of any preceding claim wherein the biocompatible electron donor is capable of donating two or more electrons.

10. The method of claim 1, wherein the biocompatible electron donor is selected from the group consisting of amine compounds; phenolic compounds; and quinones.

11. The method of claim 1, wherein the biocompatible electron donor is an amine compound comprising two nitrogen atoms each having a lone electron pair, and further comprising alkyl substituents capable of increasing the basicity of the nitrogen atoms.

12. The method of claim 1, wherein the biocompatible electron donor is N,N,N′,N′-tetramethyl-p-phenylene diamine or indocyanine green.

13. The method of claim 1, wherein the biocompatible electron donor is a phenolic compound, such as a phenol or polyphenol, in particular, a flavanol (catechins), such as epigallocatechin gallate (EGCG), epigallocatechin (EGC), epicatechin gallate (ECG) and epicatechin (EC).

14. The method of claim 1, wherein the biocompatible electron donor is a quinone, such as benzoquinone, naphthoquinone and anthraquinone.

15. The method of any preceding claim, wherein the platinum-based antineoplastic agent is selected from the group consisting of cisplatin, carboplatin, nedaplatin, oxaliplatin, satraplatin, and triplatin tetranitrate.

16. The method of any preceding claim, wherein the platinum-based antineoplastic agent is cisplatin.

17. The method of any preceding claim, wherein the platinum-based antineoplastic agent and the biocompatible electron donor are administered simultaneously or sequentially.

18. The method of any preceding claim, wherein the platinum-based antineoplastic agent and the biocompatible electron donor are administered sequentially.

19. The method of any preceding claim, wherein the biocompatible electron donor and the platinum-based antineoplastic agent are administered parenterally.

20. The method of claim 19, wherein the parenteral administration is systemic or regional.

21. The method of any preceding claim, wherein the biocompatible electron donor is administered in excess of the platinum-based antineoplastic agent.

22. The method of any preceding claim, wherein the cancer is testicular cancer, bladder cancer, cervical cancer, ovarian cancer, breast cancer, prostate cancer, head cancer, neck cancer, or lung cancer (e.g. non small cell lung cancer).

23. A synergistic combination comprising a platinum-based antineoplastic agent and a biocompatible electron donor for use in the treatment of cancer.

24. A synergistic combination comprising a platinum-based antineoplastic agent and a biocompatible electron donor for use in the manufacture of a medicament for the treatment of cancer.

25. The synergistic combination of claim 23 or 24, wherein the platinum-based antineoplastic agent and the biocompatible electron donor are as defined in any of the preceding claims, and wherein the combination is for administration in a therapeutically effective amount.

26. The synergistic combination of claim 23, 24 or 25, wherein the platinum-based antineoplastic agent is cisplatin and the biocompatible electron donor is TPMD or ICG.

27. Use of a synergistic combination of a platinum-based antineoplastic agent and a biocompatible electron donor in the treatment of cancer.

28. Use of synergistic combination of a platinum-based antineoplastic agent and a biocompatible electron donor in the manufacture of a medicament for the treatment of cancer.

29. The use of claim 27 or 28, wherein the platinum-based antineoplastic agent and the biocompatible electron donor are as defined in any of the preceding claims, and wherein the combination is for administration in a therapeutically effective amount.

30. The use of claim 27, 28 or 29, wherein the platinum-based antineoplastic agent is cisplatin and the biocompatible electron donor is TPMD or ICG

31. A kit or commercial package comprising a biocompatible electron donor and a platinum-based antineoplastic agent, together with instructions for carrying out a combination therapy for the treatment of a cancer.

32. The kit or commercial package of claim 31, wherein the biocompatible electron donor and the platinum-based antineoplastic agent are in separate pharmaceutical compositions.

33. The kit or commercial package of claim 31 or 32, wherein the platinum-based antineoplastic agent and the biocompatible electron donor are as defined in any of the preceding claims, and wherein the combination therapy is for administration in a therapeutically effective amount.

34. The kit or commercial package of claim 31, 32 or 33, wherein the platinum-based antineoplastic agent is cisplatin and the biocompatible electron donor is TPMD or ICG.

35. A biocompatible electron donor for use in combination with a platinum-based antineoplastic agent for the treatment of cancer.

36. The biocompatible electron donor of claim 34, which is TMPD or ICG.
